SEQUENCE LISTING (116 sequences)

SEQ ID NO: 1 - DNA, 30 bp, Artificial Sequence
  Description of Artificial Sequence Synthetic oligonucleotide
gaagaggaagaagaggaggaagaagaagag

SEQ ID NO: 2 - DNA, 30 bp, Artificial Sequence
  Description of Artificial Sequence Synthetic oligonucleotide
ttcctcttcttcttcctcctcttcttcctc

SEQ ID NO: 3 - DNA, 19 bp, Artificial Sequence
  Description of Artificial Sequence Synthetic oligonucleotide
ctataaaatgccggaagag

SEQ ID NO: 4 - DNA, 26 bp, Artificial Sequence
  Description of Artificial Sequence Synthetic oligonucleotide
ttcctcttccggcattttatagagct

SEQ ID NO: 5 - DNA, 25 bp, Artificial Sequence
  Description of Artificial Sequence Synthetic oligonucleotide
gaagaggagtagtcttctaactgca

SEQ ID NO: 6 - DNA, 18 bp, Artificial Sequence
  Description of Artificial Sequence Synthetic oligonucleotide
gttagaagactactcctc

SEQ ID NO: 7 - DNA, 32 bp, Artificial Sequence
  Description of Artificial Sequence Synthetic oligonucleotide
ctagaggaactagtggtaccgtagaaaaaatg

SEQ ID NO: 8 - DNA, 39 bp, Artificial Sequence
  Description of Artificial Sequence Synthetic oligonucleotide
atggtagtcgaccggcgctgcagttggatccattatttg

SEQ ID NO: 9 - DNA, 28 bp, Artificial Sequence
  Description of Artificial Sequence Synthetic oligonucleotide
gcagctgaattcgagcttggtaccgtag

SEQ ID NO: 10 - DNA, 33 bp, Artificial Sequence
  Description of Artificial Sequence Synthetic oligonucleotide
ggcatggatgagctctataaaaccatggaagag

SEQ ID NO: 11 - DNA, 26 bp, Artificial Sequence
  Description of Artificial Sequence Synthetic oligonucleotide
ctgagatgagtttttgttctagaaag

SEQ ID NO: 12 - DNA, 24 bp, Artificial Sequence
  Description of Artificial Sequence Synthetic oligonucleotide
ggaggaagaggaggaagaggaaga

SEQ ID NO: 13 - DNA, 24 bp, Artificial Sequence
  Description of Artificial Sequence Synthetic oligonucleotide
ctcctcttcctcttcctcctcttc

SEQ ID NO: 14 - DNA, 20 bp, Artificial Sequence
  Description of Artificial Sequence Synthetic oligonucleotide
ggagtagtcttctaactgca

SEQ ID NO: 15 - DNA, 12 bp, Artificial Sequence
  Description of Artificial Sequence Synthetic oligonucleotide
gttagaagacta

SEQ ID NO: 16 - DNA, 34 bp, Artificial Sequence
  Description of Artificial Sequence Synthetic oligonucleotide
gcatcagtacatgtgtgatctgcctcaaacccac

SEQ ID NO: 17 - DNA, 45 bp, Artificial Sequence
  Description of Artificial Sequence Synthetic oligonucleotide
gtcatttctagaggcggagttattattctttacttcttcttaaac

SEQ ID NO: 18 - DNA, 33 bp, Artificial Sequence
  Description of Artificial Sequence Synthetic oligonucleotide
gatcctacctgacgctttttatcgcaactctct

SEQ ID NO: 19 - DNA, 33 bp, Artificial Sequence
  Description of Artificial Sequence Synthetic oligonucleotide
cagtagagagttgcgataaaaagcgtcaggtag

SEQ ID NO: 20 - DNA, 33 bp, Artificial Sequence
  Description of Artificial Sequence Synthetic oligonucleotide
actgtttctccatacccgtttttttgggctaac

SEQ ID NO: 21 - DNA, 33 bp, Artificial Sequence
  Description of Artificial Sequence Synthetic oligonucleotide
tcctgttagcccaaaaaaacgggtatggagaaa

SEQ ID NO: 22 - DNA, 28 bp, Artificial Sequence
  Description of Artificial Sequence Synthetic oligonucleotide
aggaggttaattaaatgtgcagacctgc

SEQ ID NO: 23 - DNA, 28 bp, Artificial Sequence
  Description of Artificial Sequence Synthetic oligonucleotide
catggcaggtctgcacatttaattaacc

SEQ ID NO: 24 - DNA, 41 bp, Artificial Sequence
  Description of Artificial Sequence Synthetic oligonucleotide
gcatcattaattaaatgtgtgatctgcctcaaacccacagc

SEQ ID NO: 25 - DNA, 43 bp, Artificial Sequence
  Description of Artificial Sequence Synthetic oligonucleotide
gcattggtgcagtctagaagttattactccttacttcttaaac

SEQ ID NO: 26 - DNA, 42 bp, Artificial Sequence
  Description of Artificial Sequence Synthetic oligonucleotide
tacgacgaagacacggagtgtgatctgcctcaaacccacagc

SEQ ID NO: 27 - DNA, 44 bp, Artificial Sequence
  Description of Artificial Sequence Synthetic oligonucleotide
tacgacctgcagattattccttacttcttaaactttcttgcaag

SEQ ID NO: 28 - DNA, 47 bp, Artificial Sequence
  Description of Artificial Sequence Synthetic oligonucleotide
aggaggttaattaaatgccattgggtccagctagctctctgccacag

SEQ ID NO: 29 - DNA, 38 bp, Artificial Sequence
  Description of Artificial Sequence Synthetic oligonucleotide
tcaatggtgcagatcatgtctggatcctcgggctgggc

SEQ ID NO: 30 - DNA, 41 bp, Artificial Sequence
  Description of Artificial Sequence Synthetic oligonucleotide
gtctccgaagacgaggagactccgctgggtccagctagctc

SEQ ID NO: 31 - DNA, 43 bp, Artificial Sequence
  Description of Artificial Sequence Synthetic oligonucleotide
tcatgtatgcatgtgcagattaaggctgggcaaggtggcgtag

SEQ ID NO: 32 - DNA, 13 bp, Artificial Sequence
  Description of Artificial Sequence Synthetic oligonucleotide
ctacaaaatgccg

SEQ ID NO: 33 - DNA, 20 bp, Artificial Sequence
  Description of Artificial Sequence Synthetic oligonucleotide
ttccggcattttgtagagct

SEQ ID NO: 34 - DNA, 26 bp, Artificial Sequence
  Description of Artificial Sequence Synthetic oligonucleotide
gaataatagtctcctcctgcactgca

SEQ ID NO: 35 - DNA, 19 bp, Artificial Sequence
  Description of Artificial Sequence Synthetic oligonucleotide
gtgcaggaggagactatta

SEQ ID NO: 36 - protein, 76 aa, Artificial Sequence
  Description of Artificial Sequence Synthetic polyanionic peptide
EDEEEEEEEEEEEEEEEEEDEEEEEEEEEEEEEEEEEDEEEEEEEEEEEEEEEEEDEEEEEEEEEEEEEEEEEDEE

SEQ ID NO: 37 - protein, 14 aa, Artificial Sequence
  Description of Artificial Sequence Recognition motif
AGCKNFFWKTFTSC

SEQ ID NO: 38 - protein, 9 aa, Artificial Sequence
  Description of Artificial Sequence Recognition motif
ALNGREESP

SEQ ID NO: 39 - protein, 5 aa, Artificial Sequence
  Description of Artificial Sequence Recognition motif
CNGRC

SEQ ID NO: 40 - protein, 7 aa, Artificial Sequence
  Description of Artificial Sequence Recognition motif
ATWLPPR

SEQ ID NO: 41 - protein, 10 aa, Artificial Sequence
  Description of Artificial Sequence Recognition motif
CTTHWGFTLC

SEQ ID NO: 42 - protein, 198 aa, Artificial Sequence
  Description of Artificial Sequence Synthetic fusion protein
MAAEFELYKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEECTTHWGFTLCEE

SEQ ID NO: 43 - protein, 58 aa, Artificial Sequence
  Description of Artificial Sequence Synthetic fusion protein
MPEEEEEEEEEEEEEEEEEEEEEEEEEEEEEEEEEEEEEEEEEEEEEEEEEEEEEEEE

SEQ ID NO: 44 - protein, 186 aa, Artificial Sequence
  Description of Artificial Sequence Synthetic fusion protein
MAAEFELYKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 45 - protein, 181 aa, Artificial Sequence
  Description of Artificial Sequence Synthetic C-terminal sequence
ELYKTMEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 46 - protein, 179 aa, Artificial Sequence
  Description of Artificial Sequence Synthetic peptide
MKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 47 - protein, 11 aa, Artificial Sequence
  Description of Artificial Sequence Synthetic leader peptide sequence
MAAEFELYKMP

SEQ ID NO: 48 - protein, 177 aa, Artificial Sequence
  Description of Artificial Sequence Synthetic peptide
MPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 49 - protein, 9 aa, Artificial Sequence
  Description of Artificial Sequence Synthetic leader peptide sequence
MAAEFELYK

SEQ ID NO: 50 - protein, 187 aa, Artificial Sequence
  Description of Artificial Sequence Synthetic peptide
YKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 51 - protein, 197 aa, Artificial Sequence
  Description of Artificial Sequence Synthetic fusion protein
MAAEFELYKMPENLYFQGEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEGEEEEEEEEEEEEEEEEEEEEEEEEEEEEEEEEEEEEEEEEGCSF

SEQ ID NO: 52 - protein, 15 aa, Artificial Sequence
  Description of Artificial Sequence Synthetic peptide
EAGCKNFFWKTFTSC

SEQ ID NO: 53 - protein, 200 aa, Artificial Sequence
  Description of Artificial Sequence Synthetic fusion protein
MAAEFELYKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEAGCKNFFWKTFTSC

SEQ ID NO: 54 - DNA, 35 bp, Artificial Sequence
  Description of Artificial Sequence Synthetic oligonucleotide
cttggaagacacggaggactggggccatgaaaaac

SEQ ID NO: 55 - DNA, 34 bp, Artificial Sequence
  Description of Artificial Sequence Synthetic oligonucleotide
cttgctgcagttaactgtcctcagaagagcttgc

SEQ ID NO: 56 - protein, 176 aa, Artificial Sequence
  Description of Artificial Sequence Synthetic peptide
GEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 57 - protein, 175 aa, Artificial Sequence
  Description of Artificial Sequence Synthetic peptide
EEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 58 - protein, 350 aa, Artificial Sequence
  Description of Artificial Sequence Synthetic peptide
EEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 59 - DNA, 19 bp, Artificial Sequence
  Description of Artificial Sequence Synthetic oligonucleotide
ctataaaatgccggaagag

SEQ ID NO: 60 - protein, 6 aa, Artificial Sequence
  Description of Artificial Sequence Synthetic peptide
YKMPEE

SEQ ID NO: 61 - DNA, 26 bp, Artificial Sequence
  Description of Artificial Sequence Synthetic oligonucleotide
ttcctcttccggcattttatagagct

SEQ ID NO: 62 - DNA, 25 bp, Artificial Sequence
  Description of Artificial Sequence Synthetic oligonucleotide
gaagaggagtagtcttctaactgca

SEQ ID NO: 63 - DNA, 30 bp, Artificial Sequence; identical to SEQ ID NO: 1
  Description of Artificial Sequence Synthetic oligonucleotide

SEQ ID NO: 64 - protein, 10 aa, Artificial Sequence
  Description of Artificial Sequence Synthetic peptide
EEEEEEEEEE

SEQ ID NO: 65 - DNA, 30 bp, Artificial Sequence; identical to SEQ ID NO: 2
  Description of Artificial Sequence Synthetic oligonucleotide

SEQ ID NO: 66 - DNA, 74 bp, Artificial Sequence
  Description of Artificial Sequence Synthetic oligonucleotide
ctataaaatgccggaagaggaagaggaagaagaggaggaagaagaagaggaagaggagtagtcttctaactgca

SEQ ID NO: 67 - protein, 19 aa, Artificial Sequence
  Description of Artificial Sequence Synthetic peptide
YKMPEEEEEEEEEEEEEEE

SEQ ID NO: 68 - DNA, 74 bp, Artificial Sequence
  Description of Artificial Sequence Synthetic oligonucleotide
gttagaagactactcctcttcctcttcttcttcctcctcttcttcctcttcctcttccggcattttatagagct

SEQ ID NO: 69 - DNA, 554 bp, Artificial Sequence
  Description of Artificial Sequence Synthetic oligonucleotide
tataaaatgccggaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggagtagtcttctaactgcag

SEQ ID NO: 70 - protein, 176 aa, Artificial Sequence
  Description of Artificial Sequence Synthetic peptide
YKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 71 - DNA, 522 bp, Artificial Sequence
  Description of Artificial Sequence Synthetic oligonucleotide
gaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagag

SEQ ID NO: 72 - protein, 174 aa, Artificial Sequence
  Description of Artificial Sequence Synthetic peptide
EEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 73 - DNA, 530 bp, Artificial Sequence
  Description of Artificial Sequence Synthetic oligonucleotide
ctgcactcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttcttcttcctcctcttcttcctcttcctcttc

SEQ ID NO: 74 - DNA, 720 bp, Artificial Sequence
  Description of Artificial Sequence Synthetic oligonucleotide
ggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaagaggaggaagaggaggaagaggaaga

SEQ ID NO: 75 - protein, 240 aa, Artificial Sequence
  Description of Artificial Sequence Synthetic peptide
EEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 76 - DNA, 720 bp, Artificial Sequence
  Description of Artificial Sequence Synthetic oligonucleotide
ctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttcctcctcttcctcttcctcctcttc

SEQ ID NO: 77 - DNA, 36 bp, Artificial Sequence
  Description of Artificial Sequence Synthetic oligonucleotide
ggagtgcactacccactggggtttcactctgtgcga

SEQ ID NO: 78 - protein, 12 aa, Artificial Sequence
  Description of Artificial Sequence Synthetic peptide
ECTTHWGFTLCE

SEQ ID NO: 79 - DNA, 36 bp, Artificial Sequence
  Description of Artificial Sequence Synthetic oligonucleotide
ctcctcgcacagagtgaaaccccagtgggtagtgca

SEQ ID NO: 80 - DNA, 20 bp, Artificial Sequence
  Description of Artificial Sequence Synthetic oligonucleotide
ggagtagtcttctaactgca

SEQ ID NO: 81 - protein, 191 aa, Artificial Sequence
  Description of Artificial Sequence Synthetic peptide
YKMPEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEECTTHWGFTLCEE

SEQ ID NO: 82 - DNA, 34 bp, Artificial Sequence; identical to SEQ ID NO: 16
  Description of Artificial Sequence Synthetic oligonucleotide

SEQ ID NO: 83 - protein, 8 aa, Artificial Sequence
  Description of Artificial Sequence Synthetic peptide
MCDLPQTH

SEQ ID NO: 84 - DNA, 45 bp, Artificial Sequence
  Description of Artificial Sequence Synthetic oligonucleotide
gtttaagaagaagtaaagaataataactccgcctctagaaatgac

SEQ ID NO: 85 - protein, 6 aa, Artificial Sequence
  Description of Artificial Sequence Synthetic oligonucleotide
LRRSKE

SEQ ID NO: 86 - DNA, 25 bp, Artificial Sequence
  Description of Artificial Sequence Synthetic oligonucleotide
catgtgtgatctgcctcaaacccac

SEQ ID NO: 87 - protein, 8 aa, Artificial Sequence
  Description of Artificial Sequence Synthetic peptide
MCDLPQTH

SEQ ID NO: 88 - DNA, 18 bp, Artificial Sequence
  Description of Artificial Sequence Synthetic oligonucleotide
gtttaagaagaagtaaag

SEQ ID NO: 89 - protein, 5 aa, Artificial Sequence
  Description of Artificial Sequence Synthetic peptide
LRRSK

SEQ ID NO: 90 - DNA, 11 bp, Artificial Sequence
  Description of Artificial Sequence Synthetic oligonucleotide
ccatgtgtgat

SEQ ID NO: 91 - DNA, 15 bp, Artificial Sequence
  Description of Artificial Sequence Synthetic oligonucleotide
agaagtaaagaggaa

SEQ ID NO: 92 - protein, 5 aa, Artificial Sequence
  Description of Artificial Sequence Synthetic peptide
RSKEE

SEQ ID NO: 93 - DNA, 47 bp, Artificial Sequence; identical to SEQ ID NO: 28
  Description of Artificial Sequence Synthetic oligonucleotide

SEQ ID NO: 94 - protein, 11 aa, Artificial Sequence
  Description of Artificial Sequence Synthetic peptide
MPLGPASSLPQ

SEQ ID NO: 95 - DNA, 38 bp, Artificial Sequence
  Description of Artificial Sequence Synthetic oligonucleotide
gcccagcccgaggatccagacatgatctgcaccattga

SEQ ID NO: 96 - protein, 5 aa, Artificial Sequence
  Description of Artificial Sequence Synthetic peptide
RRSKE

SEQ ID NO: 97 - DNA, 36 bp, Artificial Sequence
  Description of Artificial Sequence Synthetic oligonucleotide
taaatgccattgggtccagctagctctctgccacag

SEQ ID NO: 98 - protein, 11 aa, Artificial Sequence
  Description of Artificial Sequence Synthetic peptide
MPLGPASSLPQ

SEQ ID NO: 99 - DNA, 38 bp, Artificial Sequence
  Description of Artificial Sequence Synthetic oligonucleotide
ctgtggcagagagctagctggacccaatggcatttaat

SEQ ID NO: 100 - DNA, 12 bp, Artificial Sequence
  Description of Artificial Sequence Synthetic oligonucleotide
gcccagcccgag

SEQ ID NO: 101 - protein, 4 aa, Artificial Sequence
  Description of Artificial Sequence Synthetic peptide
AQPE

SEQ ID NO: 102 - DNA, 10 bp, Artificial Sequence
  Description of Artificial Sequence Synthetic oligonucleotide
cgggctgggc

SEQ ID NO: 103 - DNA, 17 bp, Artificial Sequence
  Description of Artificial Sequence Synthetic oligonucleotide
ttaattaaatgccattg

SEQ ID NO: 104 - DNA, 15 bp, Artificial Sequence
  Description of Artificial Sequence Synthetic oligonucleotide
gcccagcccgaggaa

SEQ ID NO: 105 - protein, 5 aa, Artificial Sequence
  Description of Artificial Sequence Synthetic peptide
AQPEE

SEQ ID NO: 106 - DNA, 554 bp, Artificial Sequence
  Description of Artificial Sequence Synthetic oligonucleotide
tacttccagggtgaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggaagaagaggaggaagaagaagaggaagaggagtagtcttctaactgcag

SEQ ID NO: 107 - protein, 179 aa, Artificial Sequence
  Description of Artificial Sequence Synthetic peptide
YFQGEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 108 - DNA, 41 bp, Artificial Sequence; identical to SEQ ID NO: 30
  Description of Artificial Sequence Synthetic oligonucleotide

SEQ ID NO: 109 - DNA, 43 bp, Artificial Sequence
  Description of Artificial Sequence Synthetic oligonucleotide
ctacgccaccttgcccagccttaatctgcacatgcatacatga

SEQ ID NO: 110 - DNA, 27 bp, Artificial Sequence
  Description of Artificial Sequence Synthetic oligonucleotide
ggagactccgctgggtccagctagctc

SEQ ID NO: 111 - protein, 9 aa, Artificial Sequence
  Description of Artificial Sequence Synthetic peptide
ETPLGPASS

SEQ ID NO: 112 - DNA, 36 bp, Artificial Sequence
  Description of Artificial Sequence Synthetic oligonucleotide
ctacgccaccttgcccagccttaatctgcacatgca

SEQ ID NO: 113 - protein, 7 aa, Artificial Sequence
  Description of Artificial Sequence Synthetic peptide
LRHLAQP

SEQ ID NO: 114 - protein, 197 aa, Artificial Sequence
  Description of Artificial Sequence Synthetic peptide
MAAEFELYKMPENLYFQGEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEGCSF

SEQ ID NO: 115 - protein, 177 aa, Artificial Sequence; identical to SEQ ID NO: 48
  Description of Artificial Sequence Synthetic peptide

SEQ ID NO: 116 - protein, 180 aa, Artificial Sequence
  Description of Artificial Sequence Synthetic peptide
GEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEGEEEEEEEEEEEEEEEEEEEEEEEEEEEEEEEEEEEEEEEEGCSF

(19) United States
(12) Patent Application Publication
(10) Pub. No.: US 2005/0118136 A1
(43) Pub. Date: Jun. 2, 2005

(54) Recombinant production of polyanionic polymers, and uses thereof

(75) Inventors: David W. Leung, Mercer Island, WA (US); Philip A. Bergman, Mountlake Terrace, WA (US); Alan Lofquist, Kirkland, WA (US); Gregory E. Pietz, Seattle, WA (US); Christopher K. Tompkins, Bothell, WA (US); David W. Waggoner JR., Seattle, WA (US)

Correspondence Address: FOLEY AND LARDNER;SUITE 500, 3000 K STREET NW, WASHINGTON, DC, 20007, US

(73) Assignee: CELL THERAPEUTICS, INC.

(21) Appl. No.: 10/939,988

(22) Filed: Sep. 14, 2004

Related U.S. Application Data
(62) Division of application No. 10/101,487, filed on Mar. 20, 2002, now abandoned.
(60) Provisional application No. 60/277,705, filed on Mar. 21, 2001.

(51) Int. Cl. A61K 38/20; A61K 38/19; A61K 38/21; A61K 39/395; C07K 14/535; C07K 14/54

(52) U.S. Cl. 424/85.1; 424/85.7; 530/351; 424/130.1

(57) ABSTRACT

A polyanionic polymer can improve the bioactivity and water-solubility properties of a drug to which it is joined. The inventive method provides a monodispersed preparation of a recombinantly-produced polyanionic polymer that can be easily manipulated, such as lengthened. An active moiety may be chemically or recombinantly joined to a polyanionic polymer to increase its biological half-life and/or solubility. The instant invention also provides a method for targeting the delivery of a polyanionic polymer conjugate or fusion protein to a specific cell type or tissue.

### FIELD OF THE INVENTION

This application claims priority to U.S. provisional application Ser. No. 60/277,705, entitled, “Recombinant Production of Polyanionic Polymers, and Uses Thereof,” filed Mar. 21, 2001, which is incorporated herein by reference.

The instant invention relates to the recombinant synthesis of water-soluble, monodispersed, polyanionic polymers that may be purified and conjugated to a drug to enhance pharmaceutical effectiveness. Furthermore, a recombinantly-produced fusion protein of polyanionic polymer and another protein is provided by the instant invention. By genetically linking together nucleotide sequences encoding a polyanionic polymer and, for example, a therapeutic protein, the instant invention provides an efficient and precise way to modify certain properties of a protein or drug of interest.

### BACKGROUND OF THE INVENTION

The therapeutic effectiveness of a drug often depends upon its ability to dissolve in water and circulate in vivo for prolonged periods of time before being degraded or removed from the body. To this end, a drug can be chemically linked, or “conjugated,” to certain types of proteins to increase their bioavailability in vivo, as well as to enhance their solubility. For instance, the water-solubility properties of a drug can be improved by conjugating it to a polypeptide comprising amino acid residues possessing γ-carboxylic acid side chains, or to other similarly acidic side chains. The negative charges conferred by residues such as glutamate and aspartate may increase the water-solubility of drug-polypeptide conjugates. Consequently, the curative effectiveness of a drug, such as an anticancer drug, can be enhanced by conjugating it to a polypeptide that comprises many such residues. Thus, the therapeutic index of paclitaxel, an anticancer drug, may be improved when it is conjugated to the “polyanionic polymer,” poly(L-glutamic acid). See U.S. Pat. No. 5,977,163 and Li et al., Cancer Res., 58: 2404-9, 1998.

Furthermore, conjugating a therapeutic protein to a polyanionic polymer may alter the circulatory half-life of the drug. For instance, it is not unusual that a relatively small drug has a circulatory half-life of between 5 to 20 minutes. Granulocyte colony-stimulating factor (GCSF), for example, has a short biological half-life in plasma. When GCSF is chemically conjugated to polyethylene glycol, however, its plasma half-life is increased markedly (Lord et al., Clin. Cancer Res., 7: 2085-2090, 2001; van Der Auwera et al., Am. J. Hematol., 66: 245-251, 2001).

A polyanionic polymer, therefore, can change the solubility and half-life of a protein to which it is conjugated. Accordingly, the length and composition of a polyanionic polymer, and thus its molecular weight, may affect the degree to which certain properties like solubility and circulatory half-life of a conjugated protein are changed.

In this respect, polyanionic polymers are typically made using conventional chemical techniques, which can limit the size and quality of polyanionic polymer preparations. For instance, chemical methods generally cannot produce a monodispersion of polyanionic polymers larger than 10 kD. See Goud et al., J. Bone Miner. Res., 6: 781-9, 1991 and Latham, Nature Biotechnol., 17: 755-7, 1999.

Thus, chemical techniques tend to generate preparations that are non-uniform in molecular weight and size (“polydisperse”) when polyanionic polymers larger than 10 kD are required. Accordingly, it is difficult to control the specificity and quality of large molecular weight polyanionic polymers when using chemical synthesis methods.

Recombinant techniques for expressing a nucleotide encoding a polyanionic peptide do not fare any better. Only small polyanionic peptides have been expressed. For example, Zhang et al., Macromolecules, 25: 3601-03, 1992, reports of the expression of short polyanionic polymers, [H-Glu-Asp-(Glu₁₇-Asp)₄-Glu-Glu-OH], consisting of fewer than 80 amino acids. Similarly, enzymes have been fused to polyanionic peptides comprising fewer than 100 amino acids. See PCT application WO 99/33957. The difficulty in synthesizing polyglutamic acid larger than 10 kD maybe because repetitive stretches of certain amino acids, like glutamate, can form triple helices that inhibit transcription. In addition, the resemblance of polyglutamic acid coding regions made up of GAG and GAA codons to repeats of sequences that resemble the consensus of Shine-Delgarno sequence found at translation initiation sites of bacterial mRNA may inhibit translation by tying up the free 30s ribosomal subunits (Mawn et al., J Bacteriol 2002; 184: 494-502).

Thus, the field lacks a suitable method for reproducibly producing a monodispersion of a polyanionic polymer like polyglutamic acid that is at least 10 kD, or which is recombinantly fused to another protein, and which can enhance the therapeutic effectiveness, water-solubility and circulatory half-life of a drug or a protein to which it is joined.

### SUMMARY OF THE INVENTION

In view of these problems, the present invention uses recombinant DNA strategies to manufacture polyanionic polymers of specific length and molecular weight.

In one aspect, the instant invention provides a recombinantly-expressed polyanionic polymer of uniform size, generally larger than 10 kD. In another preferred embodiment, the polyanionic polymer comprises glutamate and/or aspartate amino acids.

In a preferred embodiment, the polyanionic polymer is conjugated to a drug. In a more preferred embodiment, the drug is selected from the group consisting of, but not limited to, paclitaxel, ecteinascidin 743, phthalascidin, analogs of camptothecin, analogs of epothilone, and pseudopeptides with cytostatic properties. In a preferred embodiment, an analog of camptothecin is selected from the group consisting of topotecan, aminocamptothecin, and irinotecan. In another preferred embodiment, an analog of epothilone is selected from the group consisting of epothilone A, epothilone B, pyridine epothilone B with a methyl substituent at the 4- or 5-position of the pyridine ring, desoxyepothilone A, desoxyepothilone B, epothilone D, and epothilone 12,13-desoxyepothilone F. In yet another preferred embodiment, a cytostatic pseudopeptide is selected from the group consisting of dolastatins, tubulysins, acetogenins and rapamycin.

In another embodiment, the polyanionic polymer is joined to another protein, such as to a drug, by an indirect linkage via a bifunctional spacer group. In a preferred embodiment, the preferred spacer group is relatively stable to hydrolysis, is biodegradable and is nontoxic when cleaved. In another embodiment, a spacer does not interfere with the efficacy of a polyanionic polymer-conjugate. In a further embodiment, a spacer may be an amino acid. In a preferred embodiment, an amino acid spacer may be a glycine, an alanine, a β-alanine, a glutamate, leucine, or an isoleucine. In another embodiment, a spacer may be characterized by the formula, —[NH—(CHR′)p-CO]n-, wherein R′ is a side chain of a naturally occurring amino acid, n is an integer between 1 and 10, most preferably between 1 and 3; and p is an integer between 1 and 10, most preferably between 1 and 3; hydroxyacids of the general formula —[O—(CHR′)p-CO]n-, wherein R′ is a side chain of a naturally occurring amino acid, n is an integer between 1 and 10, most preferably between 1 and 3; and p is an integer between 1 and 10, most preferably between 1 and 3 (e.g., 2-hydroxyacetic acid, 4-hydroxybutyric acid); diols, aminothiols, hydroxythiols, aminoalcohols, and combinations of these. In a preferred embodiment, a spacer is an amino acid. In a more preferred embodiment, the amino acid is a naturally occurring amino acid. In an even more preferred embodiment, the amino acid is glycine.

In another aspect of the instant invention, a therapeutic protein can be linked to a polyanionic polymer or to a spacer by any linking method that results in a physiologically cleavable bond (i.e., a bond that is cleavable by enzymatic or nonenzymatic mechanisms that pertain to conditions in a living animal organism). In one embodiment, a preferred linkage may be an ester, amide, carbamate, carbonate, acyloxyalkylether, acyloxyalkylthioether, acyloxyalkylester, acyloxyalkylamide, acyloxyalkoxycarbonyl, acyloxyalkylamine, acyloxyalkylamide, acyloxyalkylcarbamate, acyloxyalkylsulfonamide, ketal, acetal, disulfide, thioester, N-acylamide, alkoxycarbonyloxyalkyl, urea, or an N-sulfonylimidate, linkage In a preferred embodiment the linkage is either an amide or an ester linkage.

In a preferred embodiment, a low-molecular-weight chemotherapeutic agent can be conjugated to a recombinantly-produced polyanionic polymer that may be larger than 10 kD in molecular weight. In a preferred embodiment, the low molecular-weight chemotherapeutic agent is paclitaxel, camptothecin, or folate.

In one aspect of the instant invention, a fusion protein is provided that comprises a polyanionic polymer and at least one other protein. In one embodiment, the other protein may be another polyanionic polymer, a pharmaceutically active moiety, a drug, a therapeutic protein or a recognition motif sequence.

In one embodiment, the polyanionic polymer that comprises a recombinantly-produced fusion protein is larger than 10 kD. In another embodiment, the polyanionic polymer that comprises a recombinantly-produced fusion protein is not larger than 10 kD. In a further embodiment, the polyanionic fusion protein comprises a protein at either one end or at both ends of the polyanionic polymer. In another embodiment, the recombinantly-produced polyanionic fusion protein comprises a first polypeptide at the amino-terminal end of the polyanionic polypeptide and a second polypeptide at the carboxyl-terminal end of the polyanionic polypeptide. In one embodiment, the first polypeptide and the second polypeptide are the same. In another embodiment, the first polypeptide and the second polypeptide are different. In a preferred embodiment, the first polypeptide and the second polypeptide are selected from the group consisting of a targeting polypeptide and a therapeutic polypeptide.

Thus, in another embodiment, a fusion protein is expressed in a host cell that comprises a protein at the N-terminus of a recombinantly produced polyanionic polymer. In another embodiment, a fusion protein is expressed in a host cell that comprises a protein at the C-terminus of a recombinantly produced polyanionic polymer. In still another embodiment, a fusion protein is expressed in a host cell that comprises a protein at the N-terminus and at the C-terminus of a recombinantly produced polyanionic polymer. In another embodiment, the proteins that are recombinantly joined to the N- and C-termini of a polyanionic polymer are the same. In yet another embodiment proteins that are recombinantly joined to the N- and to the C-termini of a polyanionic polymer are different. In a preferred embodiment, the polyanionic polymer is recombinantly expressed glutamic acid. In another embodiment, the polyanionic polymer is recombinantly expressed aspartic acid. In a further embodiment, the polyanionic polymer is larger than 10 kD in molecular weight. In a preferred embodiment, the proteins that are recombinantly joined to a polyanionic polymer may be selected from the group consisting of a therapeutic protein and a targeting polypeptide.

In a preferred embodiment, a therapeutic protein may be one that stimulates dendritic cells. In another embodiment, a therapeutic protein may be an antigenic peptide, useful for vaccine generation.

In another preferred embodiment, a therapeutic protein or peptide is selected from the group consisting of interferon-α, interferon-β, interferon-γ, granulocyte colony stimulating factor (G-CSF), granulocyte-macrophage colony stimulating factor (GM-CSF), macrophage colony stimulating factor (M-CSF), interleukin-18, FLT3 ligand, stem cell factor, stromal cell-derived factor-1 alpha, human growth hormone, extracellular domain of tumor necrosis factor receptor, extracellular domain of tumor necrosis factor-related apoptosis-inducing ligand (TRAIL) or Apo2 ligand, extracellular domain of vascular endothelial growth factor (VEGF) receptor such as the region that includes the first 330 amino acids of the kinase domain receptor of VEGF (KDR, also known as VEGF receptor 2, the main human receptor responsible for the angiogenic activity of VEGF) or the region that includes the first 656 amino acids of VEGF receptor 1 (Flt-1), extracellular domain of transforming growth factor b type III receptor, extracellular domain of transforming growth factor b type II receptor that includes the first 159 amino acids of the receptor, herstatin that encodes the extracellular domain of HER-2/neu receptor, a secreted form of human ErbB3 receptor isoform; the secreted form of human fibroblast growth factor receptor 4 isoform, β-glucocerebrosidase, basic fibroblast growth factor, human interleukin-1 receptor antagonist, osteoprotegerin or osteoclastogenesis inhibitory factor, erythropoietin, anti-angiogenic proteins such as domain 5 region of high molecular weight kininogen or kininostatin, pigment epithelium-derived factor, vascular endothelial growth inhibitor, endostatin, restin, plasminogen kringle 1 domain, plasminogen kringle 5 domain, and angiostatin.

In another embodiment, the fusion protein may comprise a recognition, or targeting motif. In a preferred embodiment, the recognition motif is selected from the group consisting of folate, AGCKNFFWKTFTSC, ALNGREESP, CNGRC, ATWLPPR and CTTHWGFTLC.

In a more preferred embodiment, the recombinantly expressed fusion protein comprises a polyglutamic acid and a GCSF protein. In another embodiment, the polyglutamic acid is directly linked to the GCSF protein. In another embodiment at least one spacer amino acid is positioned between the polyglutamic acid and GCSF protein. In another embodiment a polyglutamic acid region may comprise at least one other amino acid, such as a spacer amino acid. In another embodiment, the polyglutamic acid has a molecular weight of more than 10 kD.

In yet another embodiment, the recombinantly expressed fusion protein comprises a polyglutamic acid and a GM-CSF protein. In another embodiment, the polyglutamic acid is directly linked to the GM-CSF protein. In another embodiment at least one spacer amino acid is positioned between the polyglutamic acid and GM-CSF protein. In another embodiment a polyglutamic acid region may comprise at least one other amino acid, such as a spacer amino acid. In another embodiment, the polyglutamic acid has a molecular weight of more than 10 kD.

In still another embodiment, the recombinantly expressed fusion protein comprises a polyglutamic acid and an interferon protein. In another embodiment, the polyglutamic acid is directly linked to the interferon protein. In another embodiment at least one spacer amino acid is positioned between the polyglutamic acid and interferon protein. In another embodiment a polyglutamic acid region may comprise at least one other amino acid, such as a spacer amino acid. In another embodiment, the polyglutamic acid has a molecular weight of more than 10 kD. In a preferred embodiment, the interferon is selected from the group consisting of, but not limited to, interferon-α, interferon-β, interferon-γ, interferon-ω, interferon-ε, interferon-κ, and hybrid interferon molecules constructed by recombinant DNA methods.

In a further embodiment, a nucleotide encoding a cell-targeting sequence that may be recombinantly joined to a nucleotide sequence encoding a polyanionic polymer is any short peptide sequence that contains an “NGR,” i.e., the amino acid sequence, asparagine-glycine-arginine. In a preferred embodiment, a cell-targeting sequence is ALNGREESP, CNGRC, CTTHWGFTLC, ATWLPPR or AGCKNFFWKTFTSC,

Another protein that may be recombinantly-linked to a polyanionic polymer is an intracellular protein that either contains or is engineered to contain a cell-penetrating peptide motif. In one embodiment, a nucleotide sequence encoding a phosphatidylehanolamine-binding protein may be recombinantly linked to a nucleotide sequence encoding a polyanionic polymer. In another embodiment, nucleotide sequences that encode tumor suppressors such as Rb, p53, PTEN, p16INK4A, p15INK4B and p14ARF, may be recombinantly linked to a polyanionic polymer of the instant invention.

In another preferred embodiment, an antibody or an antibody fragment may be recombinantly fused, or also conjugated, to a polyanionic polymer of the instant invention. To that end, in an alternative embodiment, any of the above-described proteins or peptides may also be conjugated to a polyanionic polymer of the instant invention.

In a preferred embodiment, the nucleotide sequence encoding a protein or polypeptide is operably linked to a nucleotide sequence encoding a polyanionic polypeptide in an expression cassette. In a more preferred embodiment, the nucleotide sequence encoding the polyanionic polypeptide comprises of codons encoding glutamate. In another preferred embodiment, the nucleotide sequence encoding the polyanionic polypeptide comprises of codons encoding aspartate.

In a further embodiment, a codon encoding at least one “spacer” amino acid is positioned within the nucleotide sequence encoding the polyanionic polypeptide or between the nucleotide sequence encoding the polyanionic polypeptide and the nucleotide sequence encoding a protein or polypeptide. In a preferred embodiment, the spacer amino acid is glycine, aspartate, serine, or asparagine.

In another embodiment, the expression cassette also comprises a promoter and a termination sequence, wherein the promoter functions in bacterial cells. In another aspect of the invention, the expression vector is expressed in a host cell that comprises a vector. In a preferred embodiment, the host cell expression system can be a bacterial, yeast, mammalian, or baculovirus expression system.

Thus, in one embodiment, the instant invention provides a method for expressing in a host cell a polyanionic polymer in recoverable amounts. The instant invention also contemplates the plasmid vectors and expression cassettes that are capable of expressing a polyanionic polymer fusion protein of the instant invention.

In another aspect, the instant invention provides a method for recombinantly synthesizing a monodispersed preparation of a polyanionic polymer. In one embodiment, the method comprises (1) ligating together oligonucleotides that encode anionic amino acids to form a long polynucleotide ligation product, (2) subcloning the ligation product into a vector that is capable of expressing the ligation product in a host cell, and (3) isolating the protein product of the vector, wherein the protein product is a polyanionic polymer of a specific size. In a preferred embodiment, the polyanionic polymer has a molecular weight that is larger than 10 kD.

In another aspect of the invention, a method of delivering an effective amount of a pharmaceutically active agent, a therapeutic protein or a drug to a patient in need thereof, is provided, which comprises administering to the patient a monodispersed composition of a polyanionic polymer joined, either by recombinant methods or by chemical conjugation, to a pharmaceutically active agent, a therapeutic protein or a drug. In one embodiment, the patient is a human. In another preferred embodiment, the patient is a non-human animal.

Other features, objects, and advantages of the present invention are apparent in the detailed description that follows. It should be understood, however, that the detailed description, while indicating preferred embodiments of the invention, are given by way of illustration only, not limitation. Various changes and modifications within the spirit and scope of the invention will become apparent to those skilled in the art from the detailed description.

### BRIEF DESCRIPTION OF THE DRAWINGS

FIG. 1 illustrates the location of key restriction enzyme recognition sites within plasmid clones. (A) shows the position of an Sst I restriction site just upstream of the stop codon of the nucleotide sequence encoding green fluorescent protein (GFP) in an unmodified plasmid. The restriction site Pst I is shown downstream of the 3′ end of the GFP sequence; (B) shows restriction sites introduced into a plasmid after successful insertion of a “first polyanionic-encoding nucleotide” sequence via Sst I/Pst I directional cloning. The BseR I restriction recognition sequence is encoded by the glutamate codon sequence “GAGGAG.” For this reason, a nucleotide sequence encoding a polyglutamic acid may encode several BseR I restriction sites along its length; (C) A Bbs I restriction site at the 3′ end of the first polyanionic-encoding nucleotide sequence facilitates the insertion of Bbs I/Pst I restriction fragments, such as a second polyanionic-encoding nucleotide sequence; (D) The Bbs I restriction site also faciliates the insertion at the 3′ end of the first polyanionic-encoding nucleotide sequence of a therapeutic protein or peptide or a recognition motif (not illustrated); (E) shows the insertion of a Nco I/BseR I fragment into the 5′-end of a polyanionic-encoding nucleotide sequence.

FIG. 2 shows the assembly of polyglutamic acid oligonucleotides and 5′ and 3′ adapator oligonucleotides and their insertion into a plasmid via Sst I/Pst I directional cloning.

FIG. 3 shows the purification of a polyglutamic acid product that is larger than 10 kD by anion-exchange chromatography.

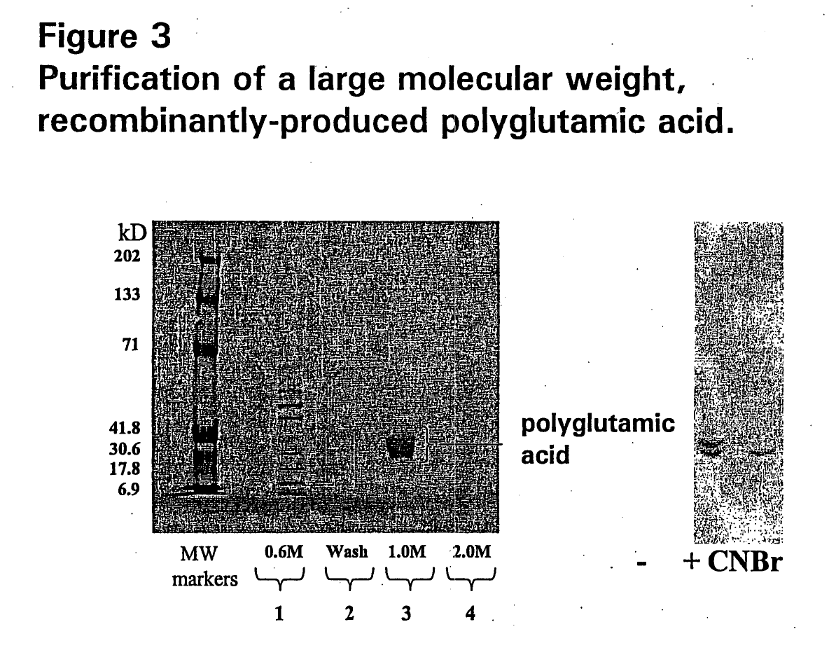

FIG. 4 shows expression of various fusion proteins of polyglutamic acid in. E. coli. Cell lysates, with or without trypsin treament, transformed with various expression plasmids and grown with or without arabinose induction were analysed by polyacrylamide gel analysis after staining with either Coomassie blue or methylene blue.

FIG. 5 shows the specific nucleotide sequences involved in the insertion of additional polyglutamic acid nucleotide sequences (a) or a specific targeting sequence (b) to the 3′ end of a polyanion-encoding nucleotide sequence, via Bbs I/Pst I directional cloning.

FIG. 6 shows the addition of interferon-α2 coding sequence to the 5′-end of a polyglutamic-encoding nucleotide sequence, via Nco I (Pci I)/BseR I(Eci I) directional cloning.

FIG. 7 shows a scheme for inserting GCSF coding sequence to the 5′-end of a polyglutamic-encoding nucleotide sequence.

FIG. 8 shows a scheme for inserting GCSF coding sequence onto the 3′ end of a polyglutamic-encoding nucleotide sequence.

### DETAILED DESCRIPTION OF THE PREFERRED EMBODIMENTS

The present invention provides a method for recombinantly producing a monodispersed preparation of a polyanionic polymer, such as a polyglutamic acid or a polyaspartic acid. The instant invention also provides a polyanionic co-polymer comprising glutamate and aspartate amino acids. The polyanionic polymer can be chemically or recombinantly joined to an active moiety. For example, a polyanionic polymer of the instant invention may be chemically conjugated to a protein or a drug. Alternatively, a nucleotide sequence encoding a polyanionic polymer can be fused to a specific gene or polynucleotide that codes for an active moiety. Thus, the instant invention also provides a recombinantly-produced polyanionic fusion protein. A polyanionic fusion protein may be conjugated to another active moiety.

The increased molecular size of the resultant polyanionic conjugate/fusion protein can lead to longer circulatory half-life and improved solubility properties of the co-joined active moiety. Kunimasa et al., J. Pharm. Pharmacol., 51: 777-82, 1999. An empirically determined effective amount of such a polyanion-drug conjugate or fusion protein can be administered to a mammal in order to treat a disease, illness or disorder. In this respect, a mammal is any animal, such as a mouse, rat, rabbit, monkey or human. A polyanionic polymer conjugate or fusion protein also may be administered to a mammal for diagnostic and testing or research purposes.

The present description uses “polymer” to denote a molecule made up of a number of repeated linked units. In this case, a “unit” may be an amino acid residue or a peptide. Thus, a polymer of the instant invention may comprise a number of repeated and linked peptides or amino acids. A “polyanion” refers to a polymer that consists essentially of negatively-charged, i.e., acidic, amino acids. As used herein, the terms, “polyanionic polymer,” “polyanionic peptide,” polyanionic polypeptide,” “polyanionic protein,” or any variation, are interchangeable. A “polyanionic fusion protein” refers to a recombinantly expressed protein that comprises a region of polyanionic polymer linked directly or indirectly to another protein.

With respect to the recombinant production of a preparation of polyanionic polymers, the term “monodispersed” refers to a population of polymers that are each approximately of the same molecular weight. In this regard, the inventive method provides a polyanionic polymer of about 1 to about 10 kD, from about 10 to about 20 kD, from about 0.20 to about 30 kD, from about 30 to about 40 kD, from about 40 to about 50 kD, from about 50 to about 60 kD, from about 60 to about 70 kD, from about 70 to about 80 kD, from about 80 to about 90 kD or from about 90 to about 100 kD in molecular weight. Preferably, a monodispersed preparation contains a population of a recombinantly-produced polyanionic polymer that is 10 kD in molecular weight. More preferably, a monodispersed preparation contains a population of a recombinantly-produced polyanionic polymer that is larger than 10 kD in molecular weight.

The instant invention, therefore, provides a recombinant method for expressing a polynucleotide that encodes a polyanionic polymer in a particular size range. Since the molecular weight of an amino acid is known, it is straightforward to estimate how long a polynucleotide sequence must be in order to produce a polyanionic polymer of a certain size. For instance, a single glutamate amino acid has a molecular weight of approximately 129 daltons. An aspartate amino acid is approximately 115 daltons. Thus, a polyanionic polymer that consists essentially of either glutamate or aspartate can be expressed that is of any desired molecular weight.

A polyanionic polymer consisting essentially of one type of amino acid, like glutamate (“E”) or aspartate (“D”) is a “homopolymer.” A protein or polypeptide that “consists essentially of” a certain amino acid is limited to the inclusion of that amino acid, as well as to amino acids that do not materially affect the basic and novel characteristics of the inventive composition. With regard to the latter, amino acids like glycine, aspartate, asparagine, or serine also can be incorporated into the inventive polymer. Thus, so long as the composition does not affect the basic and novel characteristics of the instant invention, that is, does not alter the properties of the polyanionic polymer, then that composition may be considered a component of an inventive composition that is characterized by “consists essentially of” language.

As noted above, a polyanionic homopolymer may be chemically conjugated to an active moiety. An “active moiety” refers to, but is not limited to, a drug, pharmaceutically active agent, therapeutic protein or a chemical. Any one of these active moieties may be a natural or artificial substance that is given as medicine or as part of a treatment for prophylaxis of a disease, or to lessen pain. Paclitaxel, for example, is a drug that can be conjugated to a recombinant polyanionic polymer of the present invention.

A conjugation reaction that “directly links” a drug to a polyanionic polymer typically creates bonds between a reactive group on the drug and a reactive group on the polymer. For instance, paclitaxel can be covalently linked through an ester bond to poly-L-glutamate to form a macromolecular drug delivery system. The γ-carboxyl side chain of glutamate, for example, is particularly well suited as a reactive group for this type of conjugation. For example, in conjugating interferon-α2 and polyglutamic acid, 1-ethyl-3-(3-dimethylaminopropyl)carbodiimide hydrochloride (EDC) (Pierce, Rockford, Ill.) can be used to react with one of carboxylic acid groups of polyglutamic acid to activate it and enable it to be coupled to amino groups from lysine residues in interferon-α2.

However, a drug can be conjugated to a polyanionic polymer through an indirect linkage, such as by using a bifunctional spacer group. A preferred spacer group is one that is relatively stable to hydrolysis in the circulation, is biodegradable and is nontoxic when cleaved from the conjugate. Exemplary spacers include amino acids, such as glycine, alanine, β-alanine, glutamic acid, leucine, or isoleucine. In this respect, a protein can also be conjugated to a polanionic polymer via either a histidine or a lysine directed linkage (see Example 7). Thus, Wang et al., Biochemistry, 39(35): 10634-40, 2000, indicate that the amide/ester bond links the interferon protein to another without affecting the activity of the interferon protein.

Other spacers include the chemical, —[NH—(CHR′)p-CO]n-, wherein R′ is a side chain of a naturally occurring amino acid, n is an integer between 1 and 10, most preferably between 1 and 3; and p is an integer between 1 and 10, most preferably between 1 and 3; hydroxyacids of the general formula —[O—(CHR′)p-CO]n-, wherein R′ is a side chain of a naturally occurring amino acid, n is an integer between 1 and 10, most preferably between 1 and 3; and p is an integer between 1 and 10, most preferably between 1 and 3 (e.g., 2-hydroxyacetic acid, 4-hydroxybutyric acid); diols, aminothiols, hydroxythiols, aminoalcohols, and combinations of these. Presently preferred spacers are amino acids, more preferably naturally occurring amino acids, more preferably glycine.

A spacer that can be used for such a purpose should not interfere with the efficacy of a polyanionic polymer-conjugate. Thus, a linkage moiety is used in those instances where a substance that does not have a suitable reactive group to interact with the reactive group of a polyanion. For example, a non-protein drug or a therapeutic chemical may be conjugated to a recombinant polyanionic polymer by way of a linkage moiety.

Preferably, any linking method that results in a physiologically cleavable bond by enzymatic or nonenzymatic mechanisms can be used to link a substance to a polyanionic polymer. Examples of preferred linkages include ester, amide, carbamate, carbonate, acyloxyalkylether, acyloxyalkylthioether, acyloxyalkylester, acyloxyalkylamide, acyloxyalkoxycarbonyl, acyloxyalkylamine, acyloxyalkylamide, acyloxyalkylcarbamate, acyloxyalkylsulfonamide, ketal, acetal, disulfide, thioester, N-acylamide, alkoxycarbonyloxyalkyl, urea, and N-sulfonylimidate. Most preferred at present are amide and ester linkages.

Methods for forming these linkages are well known to those skilled in synthetic organic chemistry, and can be found for example in standard texts such as ADVANCED ORGANIC CHEMISTRY, Wiley Interscience, 1992.

The present invention envisions the conjugation of a variety of proteins and drugs to a recombinantly-produced polyanionic polymer. For instance, epothilones may be conjugated to a polyanionic polymer. Examples of epothilones include but are not limited to epothilone A, epothilone B, pyridine epothilone B with a methyl substituent at the 4- or 5-position of the pyridine ring, desoxyepothilone A, desoxyepothilone B, epothilone D, and 12,13-desoxyepothilone F; pseudopeptides with cytostatic properties, such as dolastatins isolated from sea hare (Poncet, Curr. Pharm. Des., 5: 139-162, 1999) and tubulysins; and acetogenins (Liu et al., Phytochemistry, 50: 815-821, 1999; Ruprecht et al., J. Natural Products, 53, 237-278, 1990). A substance that has “cytostatic properties” is a substance that has the potential to stop the growth and development of tumor cells.

An antineoplastic agent is another active moiety that can be conjugated to a recombinantly produced polyanionic. Illustrative of antineoplastic agents are a marine natural product such as ecteinascidin 743 and its synthetic derivative, phthalascidin (Martinez et al., Proc. Nat. Acad. Sci., 96:3496-3501, 1999); analogues of camptothecin such as topotecan, aminocamptothecin or irinotecan (Verschraegen et al., Ann. NY Acad. Sci., 922: 237-246, 2000); analogues of epothilones (Altmann et al., Biochim. Biophys. Acta, 1470: M79-91, 2000).

Other conjugate candidates include poorly water soluble immunosuppressives such as rapamycin. See Simamora et al., Int. J. Pharm., 2001, 213:25-29. Camptothecin and the low-molecular-weight chemotherapeutic agent, folate, for instance, also can be conjugated to a polyanionic polymer. Reddy et al., Crit. Rev. Ther. Drug Carrier Syst., 15: 587-627, 1998.

It can be helpful to predetermine whether the activity of a protein will be affected by conjugation to a polyanionic polymer. For example, site-specific mutagenesis of two key lysine residues of interferon-α2 that are involved in conjugation was shown to have minimal effect on the antiviral or on the anti-proliferative activity of the interferon. Thus, modifications, such as conjugation reactions at these lysine positions are not likely to perturb the biological activity of interferon-α2 (Piehler et al., J. Biol. Chem., 275: 40425-33, 2000).

The instant invention also provides a method for recombinantly fusing a gene or any polynucleotide to a polyanionic polymer. A gene or polynucleotide that codes for a protein that can be conjugated to a polyanionic polymer can also be recombinantly fused to a polyanionic-encoding polynucleotide. For instance, any one member of a interferon (IFN) gene family can be recombinantly joined to a polynucleotide that codes for a polyanionic polymer. Human IFN-α and IFN-ω are encoded by gene families comprised of multiple genes. IFN-β, and IFN-γ, however, are encoded by single genes. IFN hybrid proteins have more specific antiviral activity in human cell lines than those of natural interferons. See Horisberger et al., Pharmacol. Ther., 66: 507-534, 1995 and U.S. Pat. No. 4,456,748. In general, IFNs are classified according to their molecular structure, antigenicity, and mode of induction into several isoforms. IFN-α, IFN-ω, IFN-β, IFN-ε, and IFN-κ are regarded as type I interferons, which share the same receptor and whose expression is induced by a virus. IFN-γ, however, is a type II interferon which uses a different receptor and which is induced in activated T-cells. See Whaley et al., J. Biol. Chem., 269: 10864-10868, 1994; U.S. Pat. No. 6,200,780; LaFleur et al., J. Biol. Chem., 2001. Thus, a recombinantly produced polyanionic polymer can be joined to IFN-α, IFN-ω, IFN-δ, IFN-β, IFN-ε, IFN-κ or IFN-γ.

To make a recombinantly produced polyanionic polymer, the inventive method ligates together oligonucieotides that encode either glutamate or aspartate. An oligonucleotide that encodes nine amino acid residues corresponds to half a turn of an α-helix and would impart an ordered structure to the resultant nucleic acid ligation product. Preferably, an oligonucleotide encodes at least nine anionic amino acids. However, an oligonucleotide of any length may be used according to the instant invention. An oligonucleotide may also include a “spacer” amino acid such as a serine or glycine. An oligonucleotide is preferably designed to avoid the use of repetitive DNA sequences that are known to inhibit transcription. For instance, ligated oligonucleotides containing combinations of two glutamate codons is less likely to adopt a structural configuration that impedes gene expression, than a polynucleotide made up of only one glutamate codon. Accordingly, one aspect of the present invention entails using at least two different codons to encode a particular anionic amino acid of an oligonucleotide.

Ligation products of between 200 bp and 1000 bp in size represent polynucleotides that encode large polyanionic polymers. The method of ligation is well known and is described, for instance; in Sambrook et al., MOLECULAR CLONING: A LABORATORY MANUAL, (2^{nd} ed.), section 1.53 (Cold Spring Harbor Press, 1989).

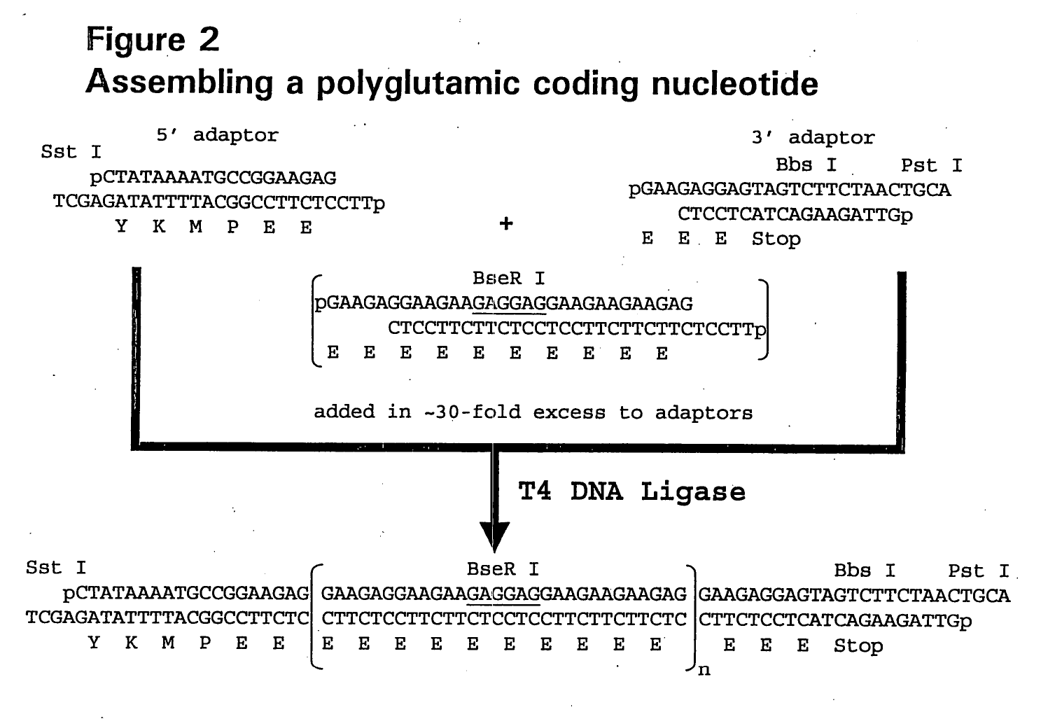

To facilitate directional cloning of the polynucleotide, the inventive methodology ligates “adaptor oligonucleotides” to the 5′ and 3′ ends of the polyanionic-encoding polynucleotide. Preferably, the adaptors contain restriction sites that are compatible with those present in an expression vector. The 3′ adaptor oligonucleotide also may comprise a stop codon to designate the end of the encoding sequence to which it is ligated (see FIG. 2). The polyanion-encoding oligonucleotides are preferably added in excess to the adaptor oligonucleotides to increase the likelihood that a long polynucleotide is generated after ligation. Thus, one polynucleotide of the instant invention comprises a number of linked oligonucleotides and is flanked at each end by restriction sites to facilitate directional cloning and also a stop codon at its 3′ end to mark the end of the coding sequence.

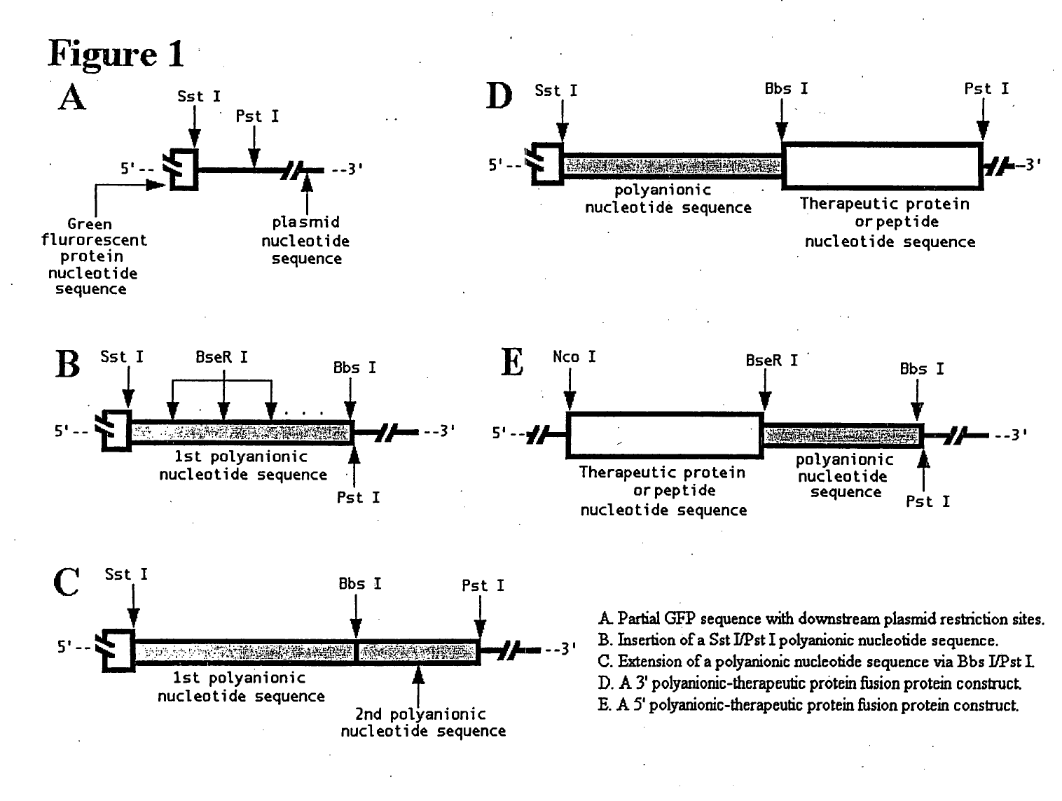

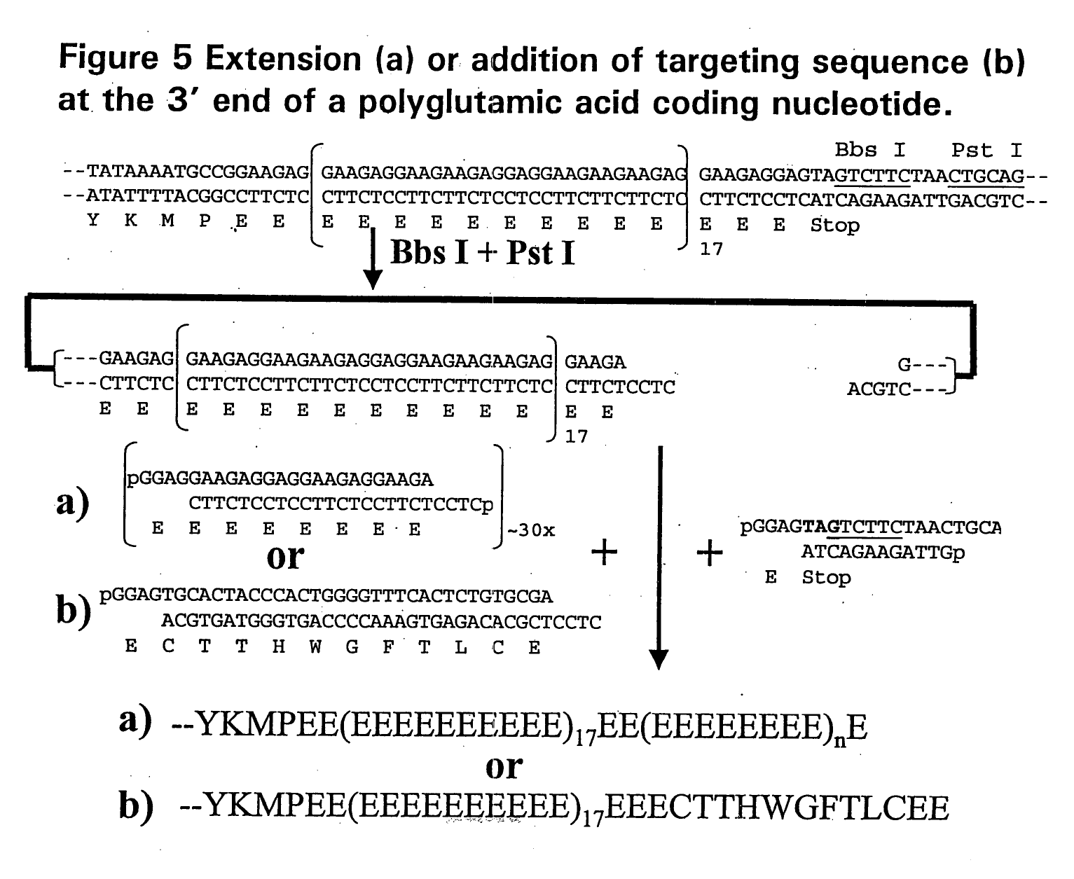

“Directional cloning” is well known to those in the art and refers to the insertion of a polynucleotide into a plasmid or vector in a specific and predefined orientation. Thus, once cloned into an expression vector, a polynucleotide sequence can be lengthened at its 3′ end or other polynucleotides inserted at its 5′ or 3′ ends. See FIG. 1(C) and FIG. 5. Such a design provides an efficient and easy way to create large polymers between 10 kD and 100 kD in size without having to perform multiple rounds of ligation, screening, and cloning. An expression vector preferably contains restriction sites upstream of a cloned polynucleotide, but downstream of regulatory elements required for expression to facilitate the insertion of a second polynucleotide 5′ to the cloned polynucleotide.

Any expression vector can be used according to the instant invention. An expression vector is typically characterized in that it contains, in operable linkage, certain elements such as a promoter, regulatory sequences, a termination sequence and the cloned polynucleotide of interest. It may also contain sequences that facilitate secretion or identification of the expressed protein.

An expression vector may contain at least one “selectable marker” or an element that permits detection of the vector in a host cell. For instance, genes that confer antibiotic resistance, such as ampicillin resistance, tetracycline resistance, chloramphenicol resistance, or kanamycin resistance can be used. A vector comprising an inducible regulatory element, such as a temperature-sensitive promoter, also can be used. Thus, expression of the polyanion-encoding polynucleotide may be induced by the addition of a certain substance, or by incubation at a certain temperature. Typically, gene expression is placed under the control of certain regulatory elements, including constitutive or inducible promoters, tissue-specific regulatory elements, and enhancers. For instance, expression of a polyglutamic acid polymer inserted into an expression vector of the instant invention, can be induced by inoculating 50 ml of culture with 0.2% arabinose for 8 hours after overnight growth. Alternatively, the regulatory elements, such as a promoter, may be a constitutive element, meaning that expression is continuous and not contingent upon certain conditions or the presence of certain substances.

The inventive methodology is not limited to the described cloning strategy. The skilled artisan may use any variety of cloning strategies to produce a vector construct that comprises a polyanionic-encoding polynucleotide that can be modified at its 5′ end and/or 3′ end.

In this respect, a nucleotide sequence or gene encoding, for example, a therapeutic protein or a recognition motif can be linked directly or indirectly to either or both ends of a cloned polynucleotide. Thus, a fusion protein may comprise a polyglutamic acid joined to a therapeutic protein at one end and a recognition motif at the other. Alternatively, a fusion protein may comprise a polyglutamic or polyaspartic acid and a therapeutic protein; or a polyglutamic acid and a recognition/targeting motif.

The polynucleotide encoding a polyanionic polymer may also be engineered to contain codons encoding a methionine (“M”) and/or a proline (“P”) amino acid at its 5′ end. Proline is unique among all amino acids in that its side-chain is bonded to the nitrogen of the amine group and to the α-carbon, to form a cyclic structure. Thus, such structures may make the polymer more resistant to aminopeptidase, an enzyme that sequentially cuts the peptide bonds in polypeptides. Additionally, proline may present steric hindrance to reduce the formation of branch-chain molecules during drug-conjugation, via interaction between the N-terminal amine and the γ-carboxyl side chains. Moreover, proline resembles the structure of pyro-glutamic acid, a cyclized form often found for the N-terminal glutamic acid. A proline can be added to the N-terminus of a polyanionic polymer or a co-polymer comprising glutamate and aspartate, for instance, to facilitate expression.

When expressed as a fusion protein, the polyanionic polymer may be of any molecular weight. Preferably, the polyanionic polymer is of sufficient size to alter certain properties, such as solubility and/or circulatory half-life of the co-joined protein.

To effect such changes in properties, the skilled artisan would know how to modify a nucleotide sequence so that it can be recombinantly linked to a nucleotide that encodes a polyanionic polymer. For example, the 3-dimensional structure of interferon-α2 shows that the C-terminal end of the molecule is a flexible coil, apparently uninvolved in any specific interaction with the rest of the protein. A truncated interferon-α2 protein, with the last five residues deleted retains all the interferon receptor-2 binding activity. Piehler et al. supra. Thus, the C-terminal end of interferon-α2 is an ideal region for inserting a polyglutamic acid sequence as it is not likely to perturb the biological activity of interferon-α2.

Similarly, the 3-dimensional structure of GCSF shows that the N-terminal end (residues 1-10) and the C-terminal end of the molecule (residues 172-173) are severely disordered and are not involved in any specific interaction with the rest of the protein (Feng et al., Biochemistry, 38: 4553-4563, 1999). A truncated GCSF protein with the first seven residues deleted retains all hematopoietic activity (Kato et al., Acta Haematol., 86: 70-78, 1991). Thus, the N-terminal end of GCSF is an ideal region for linking a polyglutamic acid sequence.

Alternatively, for secretory therapeutic proteins, a polyanionic coding nucleotide sequence may be inserted between the GCSF signal peptide coding region and the mature protein coding region to enable the secretion of the fusion protein product upon expression in cells.

The presence of polyanionic stretches, which are highly water-soluble, in a highly-expressed fusion protein also may reduce its propensity to form inclusion bodies in cells. Nevertheless, a therapeutic protein that is expressed as a fusion protein may incorrectly fold and/or be insoluble. Protein aggregates in inclusion bodies, for example, tend not to be folded correctly and therefore have less biological activity. For this reason, it may be necessary to assay the activity of a fusion protein of the present invention. To this end, one of skill in the art would know how to screen the desired protein for activity and, if necessary, how to resolubilize and re-fold the fusion protein so as to restore or improve activity. See, for instance, Misawa & Kumagai, Biopolymers, 51: 297-307, 1999.

Any nucleotide sequence can be recombinantly joined to a cloned polynucleotide of the instant invention. Exemplary of such polynucleotides includes, but is not limited to, any that encode one of the following proteins or polypeptide: interferon-α, interferon-β, interferon-γ, granulocyte colony stimulating factor (G-CSF), granulocyte-macrophage colony stimulating factor (GM-CSF), macrophage colony stimulating factor (M-CSF), interleukin-18, FLT3 ligand, stem cell factor, stromal cell-derived factor-1 alpha, human growth hormone, extracellular domain to tumor necrosis factor receptor, extracellular domain of tumor necrosis factor-related apoptosis-inducing ligand (TRAIL) or Apo2 ligand (Ashkenazi et al., J. Clin. Invest., 104: 155-62, 1999), extracellular domain of vascular endothelial growth factor (VEGF) receptor such as the region that includes the first 330 amino acids (Lu et al., J. Biol. Chem., 275: 14321-14330, 2000) of the kinase domain receptor of VEGF (KDR, also known as VEGF receptor 2, the main human receptor responsible for the angiogenic activity of VEGF) or the region that includes the first 656 amino acids of VEGF receptor 1 (Flt-1) (Miotla et al., Lab Invest., 80:1195-1205, 2000), extracellular domain of transforming growth factor b type III receptor (Bandyopadhyay et al., Cancer Res., 59: 5041-5046, 1999), extracellular domain of transforming growth factor b type II receptor that includes the first 159 amino acids of the receptor (Rowland-Goldsmith et al., Clin. Cancer Res. 7: 2931-2940, 2001), herstatin that encodes the extracellular domain of HER-2/neu receptor (Doherty et al., Proc. Natl. Acad. Sci. U.S.A., 96: 10869-10874, 1999), a secreted form of human ErbB3 receptor isoform (Lee et al., Cancer Res., 61: 4467-4473, 2001); the secreted form of human fibroblast growth factor receptor 4 isoform (Ezzat et al., Biochem. Biophys. Res. Commun., 287: 60-65, 2001), α-glucocerebrosidase, basic fibroblast growth factor, human interleukin-1 receptor antagonist, osteoprotegerin or osteoclastogenesis inhibitory factor (Yasuda et al., Endocrinology, 139: 1329-1937, 1998), erythropoietin, anti-angiogenic proteins such as pigment epithelium-derived factor (Dawson et al., Science, 285: 245-248, 1999), vascular endothelial growth inhibitor (Zhai et al., FASEB J. 13: 181-189, 1999), the domain 5 region of high molecular weight kininogen known as kininostatin (Colman et al., Blood, 95: 543-550, 2000), endostatin, restin, plasminogen kringle 1 domain, plasminogen kringle 5 domain, angiostatin and any antigenic sequence useful for vaccine generation.

A polyanionic fusion protein may also attenuate the activity of a growth factor that possesses a heparin-binding domain. A polyanionic polymer can interact ionically with proteins that contain a cluster of arginines and/or lysines, such as growth factors with heparin-binding domains. Examples of these growth factors include vascular endothelial growth factor (VEGF), basic fibroblast growth factor, heparin-binding EGF-like growth factor, pleiotrophin, midkine, hepatocyte growth factor, and platelet-derived growth factor.

A polyanionic-encoding polynucleotide may also be linked to gene that encodes a therapeutic protein that stimulates dendritic cells. Such a gene is selected from the group consisting of, but not limited to, granulocyte colony stimulating factor (G-CSF), granulocyte/macrophage colony stimulating factor (GM-CSF), macrophage colony stimulating factor (M-CSF), FLT3 ligand, stromal cell-derived factor-1 alpha, and stem cell factor.

The instant invention envisions a polyanionic fusion protein comprising GM-CSF and variants thereof. GM-CSF is a hematopoietic growth factor that stimulates proliferation and differentiation of hematopoietic progenitor cells. The polynucleotide sequence of GM-CSF is cloned into a vector that also contains a polyanion-encoding polynucleotide. Preferably, the polynucleotide of GM-CSF is recombinantly fused to the polyanion-encoding polynucleotide, such that a polyanion-GM-CSF fusion protein may be expressed in a suitable host cell. The GM-CSF coding sequence, as well as the variant forms of GM-CSF, that may be used according to the instant invention include those described in U.S. Pat. Nos. 5,393,870, 5,391,485 and 5,229,496, which are incorporated by reference herein. A “variant” refers to nucleotide or amino acid sequence that deviates from the standard nucleotide or amino acid sequence of a particular gene or protein. The terms, “isoform,” “isotype,” and “analog” also refer to “variant” forms of a nucleotide or amino acid sequence.

Similarly, “Leukine,” a recombinant human granulocyte-macrophage colony stimulating factor (rhu GM-CSF) that is produced in a yeast expression system, also may be recombinantly fused to a polyanion-encoding polynucleotide of the instant invention. The amino acid sequence of Leukine differs from the natural human GM-CSF by a substitution of leucine at position 23, and the carbohydrate moiety may be different from the native protein. Leukine is a glycoprotein of 127 amino acids characterized by 3 primary molecular species having molecular masses of 19,500, 16,800 and 15,500 daltons. Sargramostim is generally recognized as the proper name for yeast-derived rhu GM-CSF. Thus, a GM-CSF, or Leukine, or any variants thereof, may also be joined to a recombinantly produced polyanionic polymer of the instant invention.

A polyanionic fusion protein may also comprise a “recognition motif,” or a “targeting motif.” The phrase “recognition motif” denotes a targeting moiety that comprises either an amino acid sequence or a small molecule that has affinity with other proteins or biological structures. Representative cell-targeting amino acid sequences are, for example, short peptide sequences containing a NGR (asn-gly-arg) amino acid sequence, such as ALNGREESP, derived from the 9^{th} fibronectin type III repeat region, or CNGRC that shows enhanced affinity to tumor vasculature (Liu et al., J. Virol., 74: 5320-8, 2000; Arap et al., Science, 279: 377-380, 1998); a tumor targeting peptide isolated from phage display peptide libraries, CTTHWGFTLC, with a selective inhibiting activity to matrix metalloproteinase 2 (MMP2) and hence to angiogenesis and migration of tumor cells (Koivunen et al., Nature Biotechnol., 17; 768-74, 1999); a vascular endothelial growth factor (VEGF) receptor (KDR) targeting peptide, ATWLPPR, that binds KDR specifically and blocks VEGF binding to cell-displayed KDR and hence inhibits the VEGF-mediated proliferation of endothelial cells (Binetruy-Tournaire et al., EMBO J., 19:1525-1533, 2000); and the somatostatin sequence, AGCKNFFWKTFTSC, of which its receptors have been found to be overexpressed in certain tumor types (Huang, et al., Chemical Biol., 7: 453-61, 2000).

In addition to functioning as a targeting motif to tumor cells, somatostatin also has been found to inhibit tumor cell growth by binding to specific cell-surface receptors. Its potent inhibitory activity is limited, however, by its rapid enzymatic degradation and the consequently short plasma half-life (Kath & Hoffken, Recent Results Cancer Res., 153: 23-43, 2000). Hence a fusion protein comprised of a polyanionic polymer region and the somatostatin coding region may enhance its plasma half-life and its efficacy in inhibiting tumor cell growth. Possible polyanionic fusion products generated may comprise, for example, a polyanionic polymer and ALNGREESP; CNGRC; ATWLPPR; CTTHWGFTLC; or AGCKNFFWKTFTSC. FIG. 5 shows a scheme for inserting the amino acid sequence, CTTHWGFTLC, at the 3′ end of a polyglutamic acid coding region from plasmid pBDUV3B. The resultant fusion protein product would be, for instance, MAAEFELYKMP(E)₁₇₅CTTHWGFTLCEE.

Other examples of therapeutic proteins that can be expressed as fusion proteins with polyanionic polymers may include intracellular proteins that either contain or engineered with cell-penetrating peptide motifs (Lindgren et al., Trends Pharmacol. Sc., 21: 99-103, 2000). An example of such a protein is phosphatidylehanolamine-binding protein, a protein that interacts with Raf and MEK and with NF-κB-inducing kinases and acts as an inhibitor of Raf/MEK and NF-KB signal transduction activation pathways (Yeung et al., Mol. Ceil Biol., 21: 7207-7217, 2001). Other examples are proteins that code for tumor suppressor genes such as Rb, p53, p16INK4A, p15INK4B and p14ARF (Sakajiri et al., Jpn. J. Cancer Res., 92: 1048-1056, 2001).

A gene coding for an antigen for the production of vaccines (Hansson et al., Biotechnol. Appl. Biochem., 32: 95-107, 2000) can be recombinantly joined to a polyanionic polymer of the instant invention. Most of the immunogenic properties of such fusion proteins will be induced by the antigen region as the polyanionic polymer is non-immunogenic. An antibody and an antibody fragment also may considered herein as recognition motifs that can be recombinantly fused, or conjugated to a polyanionic polypeptide of the instant invention.

Any of the above-described proteins or peptides may also be conjugated to a polyanionic polymer of the instant invention. A recombinantly produced polyglutamic acid-targeting motif fusion protein may be chemically conjugated to a drug or chemical.

An expression vector comprising a polyanionic-encoding polynucleotide or a sequence encoding a polyanionic-fusion protein can be introduced by any one of a number of standard methods, such as electroporation and heat-shock treatment, into a host cell. A “host cell” is capable of transcribing and translating a cloned polynucleotide to produce a polyanionic polymer or a fusion protein, i.e., a polypeptide comprising acidic amino acids. A host cell includes but is not limited to a bacterial, yeast, mammalian, or a baculovirus cell. Similarly, expression “systems” such as bacterial, yeast, mammalian, baculovirus, and glutathione-S-transferase (GST) fusion protein expression systems can be employed to transcribe and translate the cloned polyanionic-encoding polynucleotide to produce recombinant polyanionic polymers according to the instant invention.

The instant invention envisions the expression of a polyanionic-encoding polynucleotide in a host cell under conditions that produces recoverable amounts of the resultant polyanionic polypeptide. That is, a polyanionic polymer may be expressed under conditions which produce anywhere from at least about 1 mg of polymer per liter of host cell culture.

Transformed host cells may be grown in suitable media, such as CIRCLEGROW™ (Qbiogen, Carlsbad, Calif.). Transformed host cells are harvested and lysed, preferably in a buffer that contains protease inhibitors that limit degradation after expression of the desired polynucleotide. A protease inhibitor may be leupeptin, pepstatin or aprotinin. The supernatant then may be precipitated in successively increasing concentrations of saturated ammonium sulfate. See Example 5 and also PROTEIN PURIFICATION METHODS—A PRACTICAL APPROACH, Harris et al., eds. (IRL Press, Oxford, 1989).

A polyanionic fusion protein can be purified from host cells using multi-step separations described, for instance, by Baron & Narula, Crit. Rev. Biotechnol., 10: 179-90, 1990 and Belew et al., J. Chromatogr. A., 679: 67-83, 1994. The polyanionic portion of a fusion protein can facilitate purification because the polyanion will have a high affinity for an anion-exchange column matrix. Thus, extraneous proteins isolated from host cells can be eluted from an anion exchange column using a particular concentration of NaCl. To elute polyanionic polymers of large molecular weight, a high salt concentration of NaCl may be used. See Example 5. Unprecipitated material that is soluble at high concentrations of saturated ammonium sulfate (i.e., greater than 75%) typically contains the majority of polyanionic fusion protein products.

The latter material can be dialyzed against a buffer, concentrated and chromatographed, using an anion exchange column. By eluting the column with a salt gradient from 0 M to 2.0M NaCl, the desired polymer can be obtained. Analysis of the various column fractions by colloidal Coomassie blue staining of 4-12% SDS polyacrylamide gel proves an easy way to evaluate the purity of polyanionic proteins and is a standard technique known to the skilled artisan.

The following examples are intended to illustrate, but not limit, the invention. While they are typical of those that might be used, other procedures known to those skilled in the art may be used.

### EXAMPLE 1

### Recombinant Production of Polyanionic-Encoding Polynucleotides

Oligonucleotides were ordered from MWG (High Point, N.C.) and dissolved in water at 50 pmole/ml before use. FIG. 2 shows the scheme used to assemble DNA fragments coding for polyglutamic acid.

Oligonucleotides encoding a polyglutamic acid sequence were added almost to 30-fold molar excess compared to 5′- and 3′-adaptor oligonucleotides that encode subcloning restriction sites. For instance, in addition to encoding at least one stop codon, the 3′-adaptor oligonucleotides also encode at least one asymmetric restriction enzyme recognition site, such as Bbs I, BseR I, or Bsg I (New England Biolab, Beverly, Mass.), with the cleavage sites located upstream of the recognition sites. This design allows the cleavage of the plasmid at the last codon before the stop codon of the polymer construct.

The oligonucleotide, oPG5F, was designed so that the ratio of glutamate codons, GAA to GAG. See Table 1 for oligonucleotide sequences.

6.0 μl of oligonucleotide oPG5F and 6.0 μl of oPG5R were combined with 0.2 μl of each5′-adaptor oligonucleotides, oPG6F and oPG6R; and 0.2 μl of each 3′-adaptor oligonucleotides, oPG8F and oPG8R, in a total reaction volume of 40 μl in ligation buffer in the presence of 20 units of T₄ polynucleotide kinase (New England Biolabs, Beverly, Mass.). The ligation buffer consisted of 50 mM Tris.HCl pH 7.5, 10 mM MgCl₂, 10 mM dithiothreitol, 1 mM ATP.

After incubation for 30 minutes at 37°, 400 units of T₄ DNA ligase (New England Biolabs) were added to the ligation reaction and incubated overnight at 16° C.

DNA from this reaction was precipitated according to standard techniques and digested with restriction enzymes, Sst I and Pst I, prior to fractionation and visualization of the products by standard gel electrophoresis techniques. Restriction fragments between 200 bp to 1000 bp in size were isolated for cloning into E. coli GFP fusion protein expression vectors, pBDGFP2 or pKKGFP2.

### EXAMPLE 2

### Construction of Expression Plasmids for the Synthesis of Polyanionic Polymers in E. Coli

Insertion of an Sst I-Pst I digested polynucleotide encoding anionic amino acids between the Sst I and Pst I restriction sites of either pKKGFP2 or pBDGFP2 leads to the expression, in E. coli cells, of a fusion protein comprised of a green fluorescent protein (GFP) nucleotide sequence fused to a polyanionic peptide of defined length.

(i) pKKGFP2

The plasmid pKKGFP2 was derived from the plasmids pGFPuv and pKK388-1 (Clonetech, Palo Alto, Calif.). The GFP coding region from pGFPuv was amplified in the polymerase chain reaction (PCR) to generate a product of approximately 780 bp product using oligonucleotides oGFP-2F and oGFP-2R.

This 780 bp product was digested with restriction enzymes Acc65 I and Pst I and ligated to Acc65 I and Pst I digested pKK388-1, to generate the plasmid pKKGFPuv. All restriction digests described in the instant invention were performed under conditions according to the manufacturer's instructions (New England Biolabs).

It is preferable that the construct contain a unique restriction enzyme recognition site upstream of the stop codon of GFP. To ensure that this is so, one may mutate multiple occurrences of the same restriction site sequence by PCR-based mutagenesis. For instance, the oligonucleotide, oGFP-4F, was used in a PCR reaction to mutate an N-terminal Sstl restriction enzyme recognition site (GAGCTC) to GAGCTT. See Table 1, SEQ ID NO.: 9. The GFP coding region from pKKGFPuv was amplified by PCR using oGFP-4F and oGFP-2R to generate a product of approximately 780 bp, which was then digested with restriction enzymes EcoR I and Pst I. This enabled subcloning of the restricted PCR product into the EcoR I and Pst I sites of the expression vector pKKGFPuv, generating the plasmid pKKGFP2 that has one SstI site removed. Consequently, pKKGFP2 contains only a single Sst I site upstream of the GFP stop codon. Accordingly, nucleotide sequences can be inserted at this Sst I site.

(ii) pBDGFP2

A 768 bp fragment isolated by complete Pst I and partial Nco I digestion of pKKGFP2 was inserted in between the Nco I and Pst I site of pBAD/myc-hisB (Invitrogen, Carlsbad, Calif.) to create the arabinose inducible GFP expression construct, pBDGFP2.

### EXAMPLE 3

### Expression of Cloned Polyanionic Polynucleotides in E. coli

DNA restriction mapping analysis showed that of the 200 or so cDNA clones screened, the majority contained Sst I-Pst I inserts of less than 250 bp. A single plasmid was identified with an insert of 560 bp. A silent mutation, confirmed by restriction mapping and sequencing, was found not to change the glutamic coding sequence. The 560 bp clone and another with a 200 bp insert, were chosen for expression analysis.

The 200 bp clone encodes a polyglutamic acid of 56 glutamate amino acids, corresponding to a molecular weight of approximately 7.3 kD. The 560 bp clone consists of 175 glutamic acid residues and is predicted to have a molecular weight of approximately 23 kD.

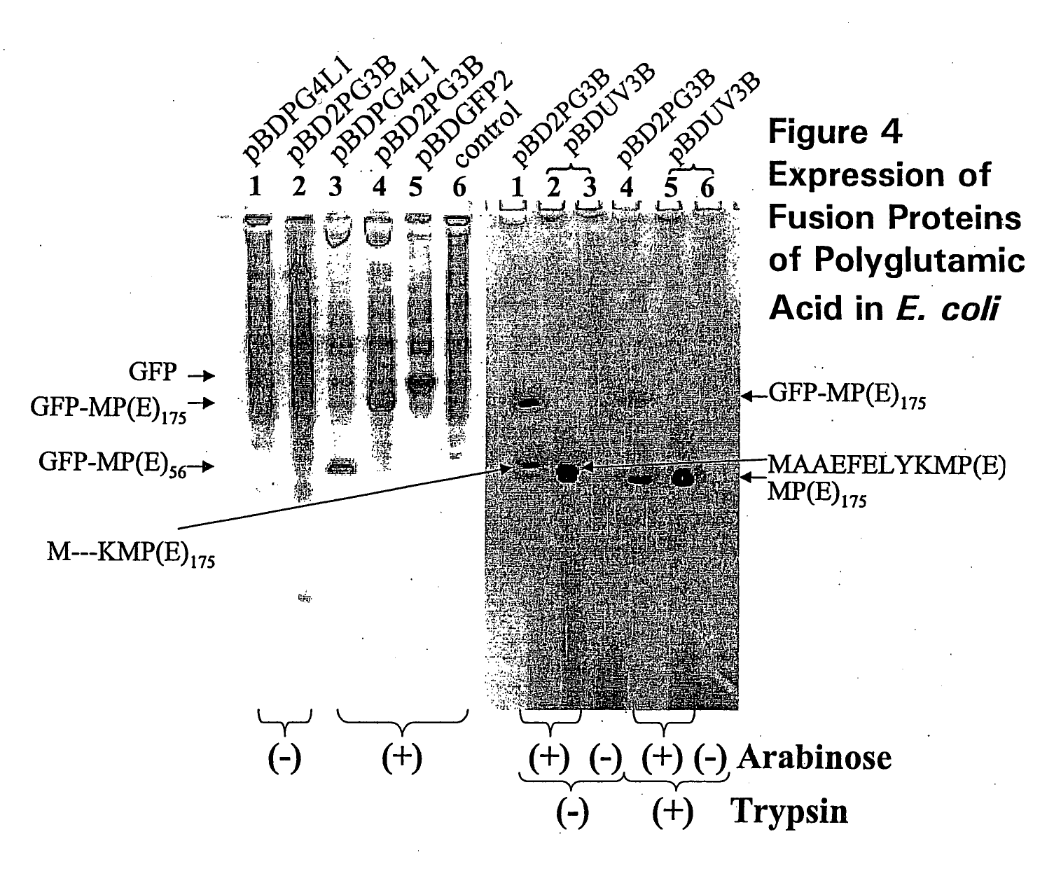

Sst I-Pst I fragments of both the 200 bp and 560 bp clones were cloned into the inducible expression vector pBDGFP2 to generate the plasmids pBDPG4L1 (200 bp clone) and pBD2PG3B (560 bp clone). After transformation of these two plasmids, along with a pBDGFP2 vector control into E. coli TOP10 strain (Invitrogen, Carlsbad, Calif.), the cells were grown in CIRCLEGROW™ (Qbiogen, Carlsbad, Calif.)±0.2% arabinose for protein analysis of cell lysates using non-denaturing acylamide gels (FIG. 4, left panel).

Cell lysates were treated with Benzonase™ nuclease (Novagen, Madison, Wis.) to remove endogenous DNA and RNA and the resultant recombinantly-produced, polyglutamic acid polymer stained with Methylene Blue.

Lanes 1 and 3 of FIG. 4 represent cells transformed with the plasmid pBDPG4L1; lanes 2 and 4 with pBD2PG3B; lane 5 with pBDGFP2; whereas lane 6 represents untransformed cells. Cells from lanes 1 and 2 were grown without arabinose; cells from lanes 3 to 6, with arabinose (FIG. 4, left panel).

Upon induction with arabinose, cells transformed with pBDPG4L1, pBD2PG3B, and pBDGFP2 (lanes 3 to 5) produced prominent protein products that are absent in uninduced cultures (lanes 1 and 2) and in the untransformed induced culture (lane 6).

Fusion protein product with 56 glutamic acid residues (lane 3, GFP-MP(E)₅₆) migrates faster than one with 175 glutamic acid residues (lane 4, GFP-MP(E)₁₇₅). Both fusion proteins migrate faster than GFP (lane 5) due to the presence of additional negative charges derived from the glutamic acids. It is expected that further increase in the chain length of polyglutamic acid would reduce the mobility that an inflection point would be reached that GFP-polyglutamic acid above a certain size would migrate more slowly than GFP.

The instant invention, therefore facilitates the expression of a polyglutamic acid comprised of a continuous stretch of 175 glutamic acids efficiently in E. coli as a fusion protein with GFP (GFP-MP(E)₁₇₅) to a level that exceeds 50% of the total E. coli cellular proteins under induced condition.

### EXAMPLE 4

### The N-Terminus of GFP is Important for Stabilizing a Recombinantly Produced Polyanionic Polymer

To determine whether polyglutamic acid can be expressed efficiently with most of GFP coding sequence absent, a 600 bp, Sst I-Pst I fragment from pBD2PG3B was isolated and ligated into Sst I- and Pst I-digested pBDGFP which removed most of the GFP, generating the plasmid pBDUV3B. This plasmid would be expected to express a fusion protein of 175 glutamic acid residues (MAAEFELYKMP(E)₁₇₅) with 10 or 11 addition amino acids at the N-terminus depending on whether the initiator methionine was removed after translation.

To remove the optional proline preceding the polyglutamic acid coding sequence in pBD2PG3B, a ˜620 bp PCR fragment was generated from template pBD2PG3B using the primers, oDP1 F and oDP1 R. This fragment was then cut with Sst I and Pst I and inserted into the vector fragment of pBD2PG3B that had been cleaved with Sst I-Pst I to generate the plasmid pBD3BNco. The plasmid pBD3BNco would be expected to express a fusion protein of GFP linked to 175 glutamates similar to that derived from pBD2PG3B. Alternatively, the proline preceding the polyglutamic acid coding sequence could be removed and the creation of an additional Nco I site at the ATG codon preceding the polyglutamic acid coding sequence incorporated. Specifically, the protein would have a C-terminal sequence of ELYK™(E)₁₇₅.

Similar to the results described in example 3, cells transformed with pBD2PG3B express a protein that has the same mobility as the GFP-MP(E)₁₇₅ product and a lower band (M—-KMP(E)₁₇₅) that may have been derived from translation initiation by AUG codons near the C-terminal end of GFP (FIG. 4, right panel, lane 1). Cells transformed with pBDUV3B produced two protein products that most likely correspond to a fusion protein of 175 glutamic acid residues (MAAEFELYKMP(E)₁₇₅) with 10 or II addition amino acids at the N-terminus, and a protein of 175 glutamic acid residues (MP(E)₁₇₅) with an additional proline and possibly a methionine at the N-terminus (FIG. 4, right panel, lane 2).

After digestion with trypsin, a protease that cleaves on the C-terminal side of lysine (K) or arginine (R), a monodispersed product corresponding to MP(E)₁₇₅ was produced (FIG. 4, right panel). Lanes 4 and 5, which represent samples from lanes 1 and 2 treated with trypsin, show the generation of a monodisperse product corresponding to MP(E)₁₇₅ as expected, with the vector pBDUV3B expressing more MP(E)₁₇₅ product. Lanes 3 and 6 represent controls to show cells grown without the inducer arabinose produce no polyglutamic acid polymer products. The expression plasmid pBD3BNco also generated products similar in size to those derived from pBD2PG3B (data not shown). It is possible, therefore, to recombinantly produce, according to the instant invention, a monodispersed polyglutamic acid product comprised of 175 glutamic acids, using the expression system described above.

The efficient production of the polyglutamatic acid fusion protein from pBDUV3B suggests that most of the GFP coding sequence is not required for high level expression of the polyglutamic fusion protein. In fact, the expression of the polyglutamic acid fusion protein is enhanced with most of the GFP coding sequence removed. However, the leader peptide sequence MAAEFELYKMP that precedes the M(P)_{O/1}(E)₁₇₅ coding sequence in plasmid pBDUV3B, is critical for high level expression of the polyglutamic acid fusion protein in E. coli, since constructs lacking MAAEFELYKMP produce no methylene-blue stainable product of M(P)_{O/1}(E)₁₇₅ on polyacrylamide gels. Instead, those constructs produced increased amounts of diffused products at bottom of the gels (data not shown). These data indicate that the MAAEFELYKMP leader peptide is important for the stability of the polygiutamic acid fusion protein product.

### EXAMPLE 5

### Purification of a Polyanionic Polymer

A frozen pellet of bacteria (from 50 ml culture that had been induced for 5 hours with 0.2% arabinose after overnight growth, followed by a 1:8 dilution with CIRCLEGROW™ containing 4% glycerol and continuous growth for 3 hours (Qbiogen, Carlsbad, Calif.) media) was thawed and solublized in 5 ml of lysis buffer (10 mM Tris, pH 7.7, 1 mM EDTA, 0.1% TX-100, 0.2 mg/ml Lysozyme, 1 mM AEBSF, 1 mM Benzamidine, μg/ml Leupeptin, 1 μg/ml Pepstatin A, 1 μg/ml Aprotinin, 1 μg/ml E-64).

The mixture was vortexed vigorously and sonicated twice on ice at power setting of 1.5, with continuous duty for 60 s (Branson Sonifier, microtip). Benzonase™ nuclease (Novagen, Madison, Wis.) was added to a final concentration of 50 U/ml, and the mixture allowed to stand at room temperature for 60 minutes.

The sample was then centrifuged 109,000×g for 60 min at 4° C. The soluble material in the supernatant was precipitated in successively increasing concentrations (0-40%, 40-50% and 50-75%) of saturated ammonium sulfate. The unprecipitated material soluble at >75% saturated ammonium sulfate was found to contain the majority of the polyglutamic acid fusion protein products.

This unprecipitated material was dialyzed to equilibrium against 10 mM Tris, pH 7.7, concentrated using Centricon filters (Millipore, Bedford, Mass.), and chromatographed on a Mono Q column (anion exchange) using an FPLC apparatus (Amersham Pharmacia, Piscataway, N.J.). The column was eluted with a salt gradient from 0 M to 2.0M NaCl. The various column fractions were analysed by 4-12% SDS polyacrylamide gel (Invitrogen, Carlsbad, Calif.) followed by colloidal Coomassie Blue staining (Neuhoff et al., Electrophoresis, 1988, 9: 255-62).

All the extraneous proteins from E. coli were found to be eluted at the early fractions, whereas the ˜23 kD polyglutamic acid fusion protein products were found to be eluted at later fractions with the higher salt concentration. As no other proteins can be detected by colloidal Coomassie Blue staining in this higher salt eluate, these results suggest that polyglutamic acid fusion protein products can be readily purified from E. coli extracts using a 75% (NH₄)₂SO₄ precipitation step to remove certain extraneous proteins followed by high salt elution from anion-exchange chromatography.

The Mono Q-purified polyglutamic acid fusion protein product exhibited a doublet banding pattern on polyacrylamide gel. To determine whether this doublet pattern could be attributed to the presence of two possible translation start sites in the coding sequence, generating the products MAAEFELYKMP(E)₁₇₅ and MP(E)₁₇₅, the purified material was incubated with cyanogen bromide under standard hydrolytic conditions (Epstein et al., J. Biol. Chem., 250: 9304-12, 1975) and then evaluated on polyacrylamide gel. CNBr treatment converted the doublet into a single band. Thus, the presence or the absence of the 9 amino acid leader sequence (MAAEFELYK) accounts for the slightly different mobility of the polyglutamic acid protein on polyacrylamide gel. This interpretation is consistent with the results of proteolysis experiments using trypsin as well (example 4 and FIG. 4, right panel). Resistance of the protein product to complete degradation by trypsin or CNBr also is consistent with a protein made of polyglutamate.

After purification of the fusion protein, the GFP portion or the leader peptide portion can be removed by digesting the fusion protein with trypsin or through CNBr treatment, as the polyglutamic acid region does not contain any internal lysine, arginine, or methionine, and therefore would be resistant to trypsin or CNBr treatment.

### EXAMPLE 6

### Extending the Length of a Polyanionic Polymer

To obviate the need to screen hundreds of clones for putatively long stretches of a polyanionic-encoding polynucleotide, a scheme was developed pursuant to the present invention, for extending an extant cDNA clone, such as the one described above, that contains the coding sequence for 175 glutamates.

To this end, plasmid pBD2PG3B or pBDUV3B was digested with Bbs I and Pst I. Since the 3′-adaptor oligonucleotide is designed with unique restriction sites, it is possible to introduce other polynucleotides at that site. For instance, the unique asymmetric restriction enzyme recognition site for Bbs I, (5′-GTCTTC) in the 3′-adaptor oligonucleotide overlaps the last nucleotide of the TAG stop codon for the polyglutamic acid fusion protein. The Bbs I cleavage site is located just upstream of its recognition site. Thus, a plasmid can be digested at the codon just prior to the stop codon of the polynucleotide insert than encodes the desired polyanion.

Accordingly, nucleotides encoding polyanionic amino acids can be fused on to the end of the originally cloned polyglutamate-encoding insert to facilitate lengthening of the polyanionic polymer at the carboxyl-terminus. This newly added nucleotide fragment may contain a different arrangement of glutamate or aspartate or other amino acid codons, so as to minimize the detrimental effect of long stretches of repeat sequences upon expression.

Accordingly, 61 μl of oligonucleotide, oPG9F, 6 μl of oligonucleotide oPG9R, 0.2 μl of oligonucleotide oPG10F and 0.2 μL of oligonucleotide oPG11R were mixed in a total volume of 40 μl in ligation buffer (50 mM Tris.HCl pH 7.5, 10 mM MgCl₂, 10 mM dithiothreitol, 1 mM ATP) and 20 units of T₄ polynucleotide kinase (New England Biolabs, Beverly, Mass.). After 30 min at 37° C., 400 units of T₄ DNA ligase (New England Biolabs) were added and the reaction was incubated at 16° overnight. The DNA from the sample was precipitated with 2.5 volume of EtOH after adjusting the sample to pH. 6 with 0.3M NaOAc. The ligated DNA was then cut with Pst I prior to fractionation of the products by gel electrophoresis. Fragments between 150 bp to 1000 bp were isolated for cloning in between the Bbs I and Pst I sites of plasmid pBD2PG3B or pBDUV3B for the production of fusion proteins with the sequences—YKMPEE(EEEEEEEEEE)₁₇EE(EEEEEEEE)ₙE at the carboxyl termini.

A clone with the longest insert, pBD3B-7, was chosen for further study. DNA sequence analysis showed the insert encoded 271 glutamic acids, corresponding to a molecular weight of 35.0 kD. Cells transformed with pBD3B-7 produced an upper methylene blue-stained band corresponding to the GFP-polyglutamic acid and a lower band from translation initiation using AUG codons found near the C-terminal end of GFP.

It is therefore possible to recombinantly produce a monodisperse, polyglutamic acid product in E. coli comprised of 271 glutamic acids using the inventive method. Because the unique restriction sites, Bbs I and Pst I, near the 3′ end of the polymers are retained after each step of extension, one can use this inventive method repeatedly, and in so doing, extend the length of the encoding sequence and thus obtain polyanionic polymers of larger molecule weight.

One skilled in the art can employ this methodology to add other nucleotide sequences to the 3′ end of the cloned insert. Such sequences include but are not limited to recognition motifs, signaling sequences, and therapeutic proteins, as described above.

### EXAMPLE 7

### Recombinant Production of Therapeutic-Polyanionic Fusion Proteins

A cell-targeting motif or therapeutic protein can be fused to the amino-terminal end of a cloned insert encoding a polyanionic polymer. In this case, the plasmid is digested with restriction sites located upstream of the cloned insert and within the cloned insert. For example, in the present invention, an Nco I site within the plasmid is used, as is the asymmetric BseR I restriction site found within the sequence encoding polyglutamic acid. A double stranded synthetic DNA with compatible Nco I and compatible BseR I cohesive ends that encode cell-specific recognition motifs can be inserted into a plasmid vector, such as pBD3B-7, pBD2PG3B, pBDUV3B, or pBD3BNco, that was digested to completion with Nco I and partially digested with BseR I. A partial digest of the vector with BseR I is required as there would exist multiple BseR I restriction sites within the polyglutamic acid coding region. Clones with long polyglutamic acid inserts can be obtained by screening various clones generated by restriction mapping to find ones where the cleavage occurred near the N-terminal side of the polyglutamic acid coding region.

A number of different polynucleotides can be inserted alongside a cloned polyanionic polymer, such that upon expression, a fusion product is produced. For instance, interferon can be recombinantly fused to a polyglutamic acid, as can granular colony stimulating factor and somatostatin. The following examples show that such fusion products can be produced using the inventive methodology and that the resultant expression products are viable.

(i) Recombinant Production of an N-Terminal Interferon-Polyanionic Polymer fusion protein

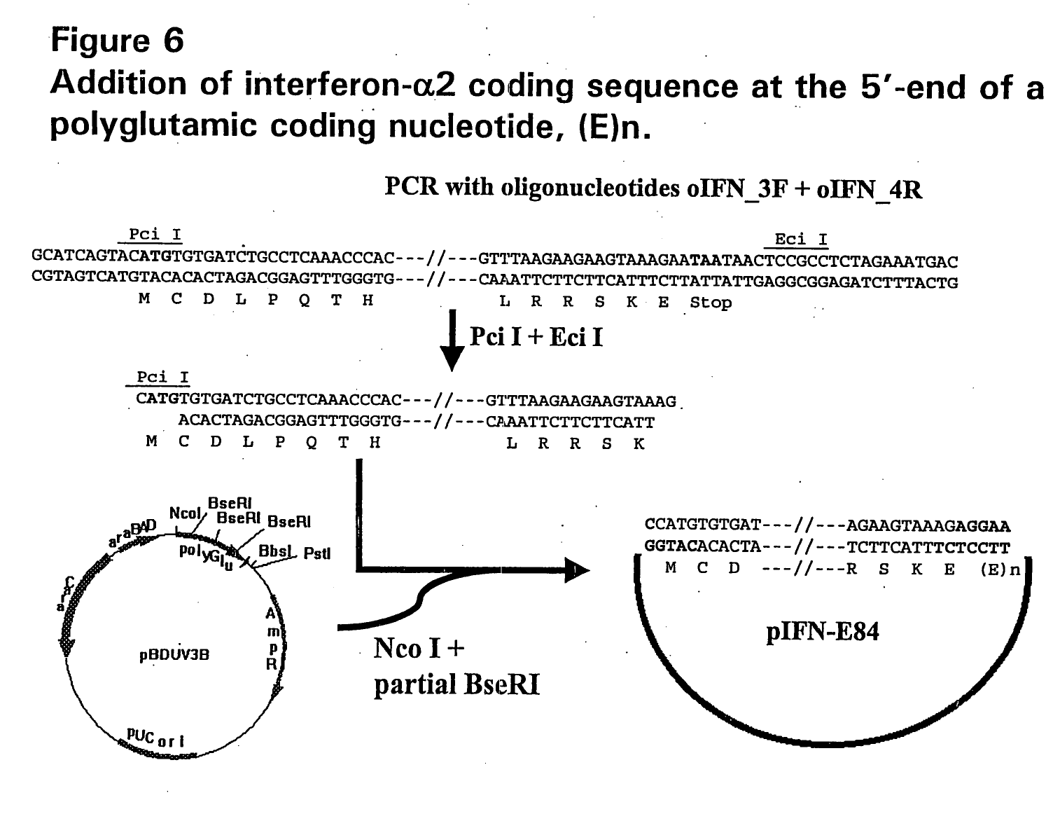

Oligonucleotides oIFN-3F and oIFN-4R were used to amplify the mature coding sequence of mature human interferon-α2 from human genomic DNA or human cDNA library by PCR. oIFN-3F was designed to contain a Pci I site that overlaps the ATG codon of the amplified human interferon-α2. Similarly, oIFN-4R contained an Eci I site, which was introduced downstream of the interferon stop codon such that its cleavage site spans the last nucleotide of the penultimate codon and the first nucleotide of the last codon of the coding sequence of human interferon-α2. See FIG. 6.

The ˜540 bp PCR fragment thus generated then was cleaved with Pci I and Eci I. The resultant fragment of ˜505 bp was isolated by gel electrophoresis. The ˜505 bp fragment has Pci I and Eci I cohesive ends that are compatible with Nco I and BseR I digested ends, respectively. Thus, the 505 bp interferon restriction fragment was inserted into the plasmid pBDUV3B, which had been digested to completion with Nco I and partially digested with BseR I. The resultant mature human interferon-α2 would contain, upon expression therefore, a polyglutamic acid at its carboxyl end.

A cDNA, pIFN-E84, expressing a fusion protein comprised of the mature coding sequence of human interferon-α2 and a polyanionic tail of 84 glutamic acids was chosen for further study. The ˜525 bp Pci I-Xba I fragment was inserted into the plasmid pBDUV3B, which had been digested to completion with Nco I and Xba I, to generate the plasmid pBdIFN [symbol] 2 for the expression of mature human interferon-α2.

To facilitate simpler methods of in-frame insertion of various genes upstream of the polyglutamic acid coding region without the requirement for partial digest with BseR I, the plasmid pBD3Bnco was modified to generate pBDRPBBN. pBDRPBBN has a Pac I restriction site just downstream of the ribosome binding site for translation of the fusion protein, a Bsg I and a BspM I restriction recognition sites upstream of the polyglutamic acid coding region in such a way that their cleavage sites would occur within the polyglutamic acid coding region. Specifically, the oligonucleotides oMCS1 F, oMCS1R, oMCS2F, oMCS2R, oMCS3F, and oMCS3R were annealed and ligated to the 4535 bp BamH I-Nco I vector fragment derived from pBD3Bnco to generate pBDRPBBN. With the availability of pBDRPBBN, cDNA fragments generated by PCR with a Pac I restriction site engineered upstream of the ATG translation initiator codon and a Bsg I or a BspM I restriction recognition site engineered downstream of the 3′-end of the coding sequence with the stop codon removed can be inserted into pBDRPBBN vector that has been cleaved with Pac I and either Bsg I or BspM I for the expression of fusion proteins with a defined numbered of glutamic acid residues at the carboxyl-terminal end.

Specifically, mature human interferon-α2 coding sequence was amplified from human genomic DNA using the PCR primers oIFNMCS-3F and oIFNMCS-2R to generate a 540 bp fragment. The 540 bp fragment was cleaved with Pac I and Bsg I to generate cohesive ends that can be ligated with a vector fragment derived from cleaving the plasmid pBDRPBBN with Pac I and Bsg I to generate the plasmid pIFN175E for the expression of a fusion protein, IFNa2-E173, comprised of mature IFN-α2 sequence with a tail of 173 glutamic acids on the carboxyl terminal side.

The availability of expression constructs, such as pIFN175E or pTEV175IF, for the synthesis of interferon fusion proteins with nolvalutamic acid either on the carboxyl- or the amino-terminal side of interferon would also facilitate construction of new expression vectors. Examples of these new vectors can express interferon fusion proteins with polyglutamic acid on both the carboxyl- and the amino-terminal side of interferon, and express tandem interferon fusion proteins with a polyglutamic acid sequence in between. Using a unique restriction site, PpuM I, present with the coding region of IFNα2, an 1020 bp PpuM IXba I fragment was isolated from pIFN175E and subsequently inserted into a 4650 bp PpuM I-Xba I vector fragment derived from pTEV175IF to generate the plasmid pE-INF-E for the expression of an interferon fusion protein with polyglutamic acid on both the carboxyl- and the amino-terminal ends. Using a similar method based on extension through the Bbs I and Pst I sites, the same 530 bp fragment of mature human interferon-α2 coding sequence amplified from human genomic DNA using the PCR primers oIFNBB-1 F and oIFNPS-2R was cleaved with Bbs I and Pst I to generate cohesive ends that can be ligated into a vector fragment derived from cleaving the plasmid pIFN175E with Bbs I and Pst I to generate the plasmid pIF-E-IF for the expression of a tandem interferon fusion protein with a polyglutamic acid sequence in between.

(ii) Recombinant Production of an N-Terminal GCSF-Polyanionic Polymer Fusion Protein

In similar fashion, PCR products coding for GCSF protein with compatible Nco I and compatible BseR I cohesive ends can be generated.

Specifically, mature human GCSF coding sequence was amplified usingthe PCR primers oGCSF-3F and oGCSF-3R to generate a 560 bp fragment.

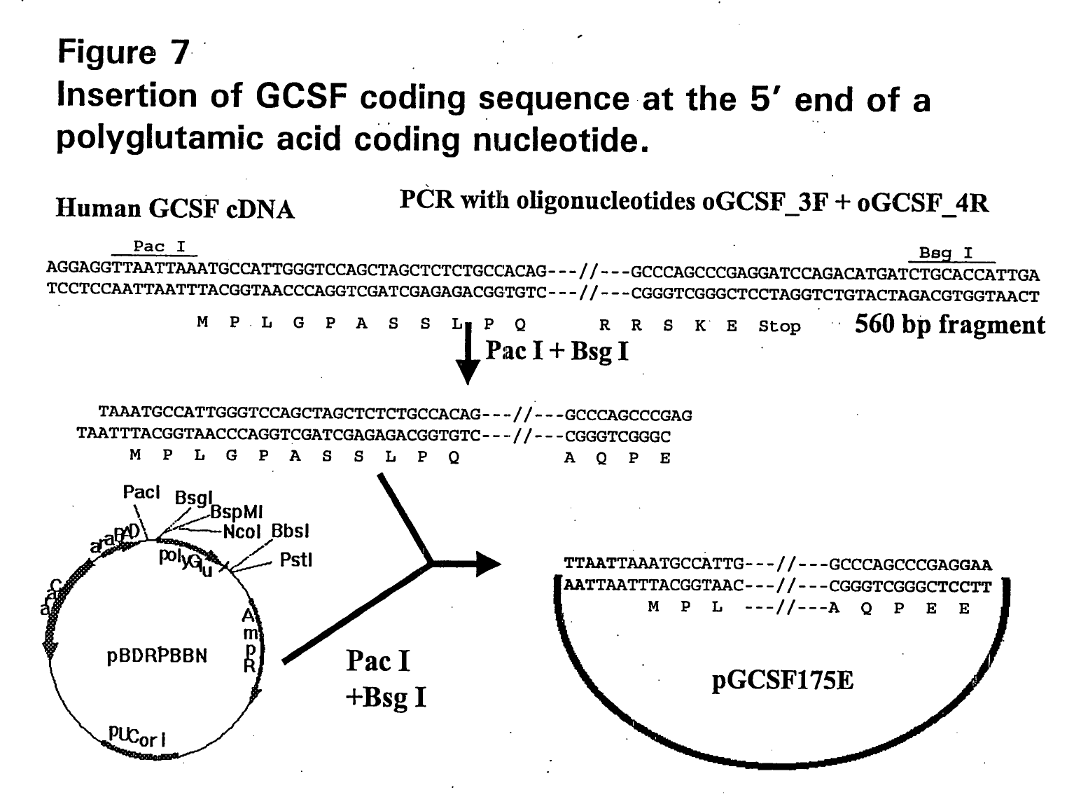

The 560 bp fragment was cleaved with Pac I and Bsg I and ligated into Pac I and Bsg I digested pBDRPBBN to generate the modified GCSF molecule, pGCSF175E (FIG. 7). This plasmid can be used to express GCSF-polyglutamic acid fusion protein, comprised of mature GCSF sequence with a tail of 174 glutamic acids on the carboxyl terminal side.

(iii) Recombinant Production of a C-Terminal GCSF-Polyanionic Polymer Fusion Protein

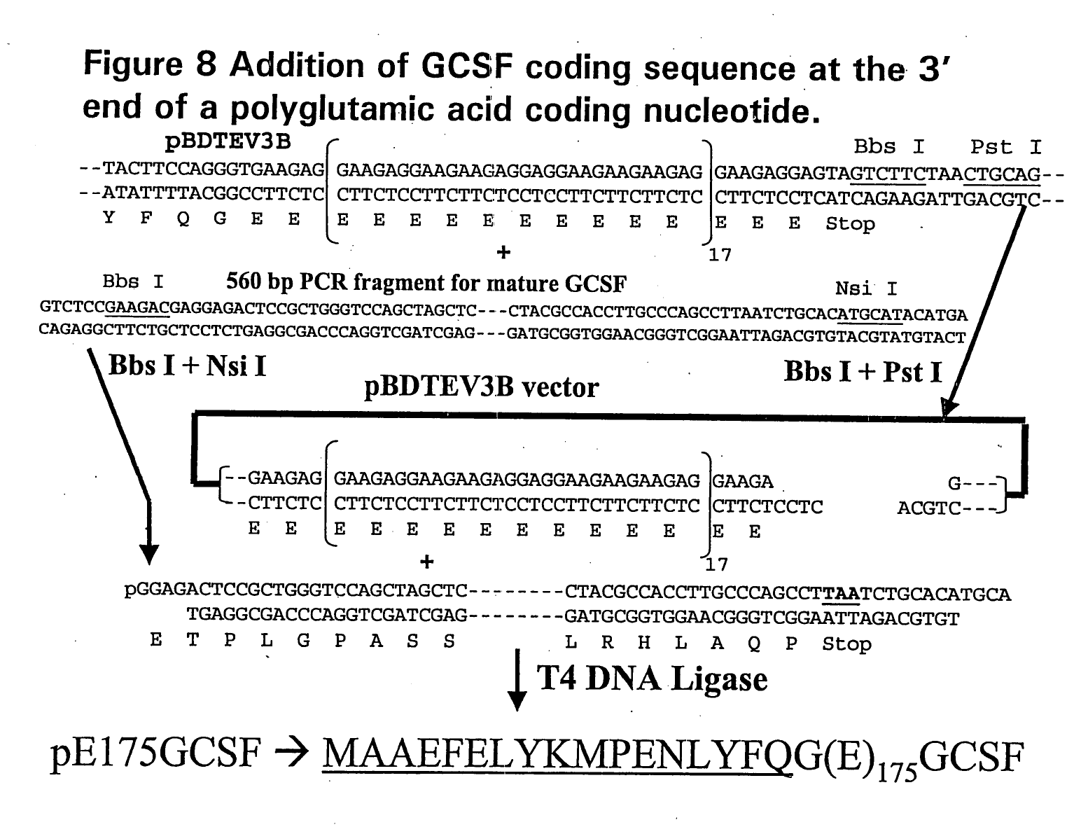

The mature human GCSF coding sequence was amplified from a GCSF cDNA clone described in U.S. Pat. No. 6,171,824 using the PCR primers oGCSF_{—}4F and oGCSF_{—}4R to generate a 560 bp fragment. The 560 bp fragment was cleaved with Bbs I and Nsi I to generate a 540 bp fragment that was ligated into with a Bbs I and Pst I digested, pBDTEV3B to generate pE175GCSF. See FIG. 8. Accordingly, the resultant recombinantly-produced fusion protein comprises MAAEFELYKMPENLYFQG(E)₁₃₄G(E)₄₀GCSF, which represents a leader peptide with a TEV protease recognition sequence, polyglutamic acid and the mature sequence of GCSF. The presence of the TEV protease sequence allows cleavage of the fusion protein to generate the peptide, G(E)134G(E)40GCSF after appropriate TEV protease (Invitrogen, Carlsbad, Calif.) treatment.

Western blot analysis of E. coli Top10 lysates transformed with the plasmid pE175GCSF showed that the polyglutamic acid-GCSF fusion protein was expressed as a doublet of approximately 42 kD. The doublet is mostly likely due to presence of in E. coli of a protease that can also cleave the recognition sequence of TEV protease (Invitrogen, Carlsbad, Calif.), as addition of TEV protease can convert the doublet into a single band corresponding to the faster moving band of the doublet (data not shown). Analysis of Top10 strain (Invitrogen, Carlsbad, Calif.) E. coli cells after lysing with BugBuster™ (Novagen, Madison. WI) followed by fractionation into the pellet and supernatant fractions shows most of the polyglutamic acid-GCSF fusion proteins produced are found in the supernatant or the soluble fraction. GCSF produced in E. coli is largely found in the pellet fraction known as inclusion bodies (Lu et al., Protein Expr Purif 1993, 4: 465-472). Such protein aggregates in inclusion bodies tend not to be folded correctly and therefore require extensive refolding process to restore their biological activity and solubility. The predominant presence of polyglutamic acid-GCSF fusion proteins in the soluble fraction would confirm the idea that polyanionic stretches, which are highly water-soluble, in a fusion protein may have the advantage to reduce its propensity to form inclusion bodies in cells.

(iv) Recombinant Production of a Somatostatin-Polyanionic Polymer Fusion Protein

The unique Bbs I site and Pst I site in the plasmid pBD2PG3B or pBDUV3B can be used for insertion of double stranded synthetic DNAs with compatible Bbs I and/or Pst I cohesive ends that encode somatostatin coding sequence.

The possible products generated may contain the amino acid sequence (E)nAGCKNFFWKTFTSC at the carboxyl-terminal end. An example of a scheme for inserting synthetic DNA fragments coding for the amino acid sequence of somatostatin, AGCKNFFWKTFTSC, onto the C-terminal side of the polyglutamic acid coding region from plasmid pBDUV3B for the expression of the fusion protein product MAAEFELYKMP(E)175 AGCKNFFWKTFTSC using the expression plasmid npBDPGSOM is shown.

A 28 aa precursor form of somatostatin has also been found to be active. This sequence can also be used in lieu of the 14 aa somatostatin form described here. The somatostatin sequence(s) can also be inserted on the N-terminal of PG or on both the N-terminal and C-terminal of PG.

(v) Recombinant Production of a Polyglutamic Acid-Kininogen 5′ Domain Fusion Protein

An example of an expression plasmid that can be used to express a polyglutamic acid-kininogen 5′ domain is described herein. The oligonucleotides oKinD5F1: 5′-CTTGGAAGAC ACGGAGGACT GGGGCCATGA AAAAC-3′ and oKinD5R2: 5′-CTTGCTGCAG TTAACTGTCC TCAGAAGAGC TTGC-3′ were used to amplified the coding sequence of corresponding to domain 5 of high molecular weight kininogen by PCR using either human genomic DNA or human cDNA library as template. The 340 bp PCR fragment generated was comprised of the coding region corresponding to amino acids 412-513 of high molecular weight kininogen with an in-frame stop codon downstream and was flanked by Bbs I and Pst I sites. The 340 bp DNA was then cut with Bbs I and Pst I prior to isolation of the 330 bp product by gel electrophoresis. The isolated fragment was then inserted in between the Bbs I and Pst I sites of plasmid pBDUV3B for the production of polyglutamic acid-kininostatin fusion protein.

### EXAMPLE 8

### Assaying the Biological Activity of a Recombinantly-Produced, Polyanionic Fusion Protein

(i) Assaying the Activity of a Recombinantly Produced Interferon-Polyanionic Polymer

A method to determine the potency of interferons is to assay their anti-proliferative response on Daudi cells (Piehler et al., J. Biol. Chem., 2000, 275: 40425-33). Samples of Origami strain (Novagen, Madison Wis.) E. coli expressing IFNa2-E84 from pIFN-E84 (IFNE84), expressing IFNα2 from pBdIFNα2 (IFN), expressing GFP from pBDGFP2, and expressing MAAEFELYKMP(E)₁₇₅ from pBDUV3B (UV3B) were dissolved in 8M guanidine hydrochloride and then diluted 10 fold with RPMI growth medium. Serial dilutions of these samples were then applied to Daudi cells plated previously on 96-well plates. The effect of samples on Daudi cells proliferation was assessed using the Alamar Blue assay (O'Brien et al., Eur J Biochem 2000; 267: 5421-5426). The toxic effect of guanidine hydrochloride in the samples is negligible after serial dilution #3, as control extracts expressing either GFP or MAAEFELYKMP(E)₁₇₅ have minimal effect on Daudi cell proliferation from serial dilution #3 to #12. On the other hand, E. coli extracts expressing IFNa2-E84 or IFNa2 inhibit the Daudi cell proliferation significantly from serial dilution #3 to #10, suggesting that the fusion protein IFNa2-E84 is as active as mature IFNα2 and that the addition of polyglutamic acid to the carboxyl-terminal end of interferon does not impair the biological activity of interferon. Similarly, constructs expressing mature IFN-α2 sequence with a tail of 173 glutamic acids on the carboxyl terminal side from plasmid pIFN175E or expressing G(E) 175IFN-α2 from plasmid pTEV175IF with polyglutamic acid linked to the amino-terminal end of interferon are also active in the Daudi cell anti-proliferation assays (data not shown).

Interferon can inhibit the proliferation of many cell types through the activation of transcription factor Stat1 by the Janus kinase signal transducers (Bromberg et al., Proc Natl Acad Sci USA 1996; 93: 7673-7678). Accordingly, another method of evaluating the biological activities of the interferon polyglutamic acid fusion proteins is to assess their capability of phosphorylating Stat1 in cells. Stat1 phosphorylation assays can be performed by Western analysis on adding several E. coli extracts expressing IFNα2-polyglutamic acid constructs onto Daudi cells. E. coli cells grown and induced from 5 ml culture was resuspended 100 μl in 8M guanidine hydrochloride and then diluted 40-fold with RPMI growth medium. 100 μl sample aliquots were then added onto Daudi cells plated in T-25 flasks at 750,000 cells per flask. After 20 minutes, Daudi cell extracts were prepared for Western analysis using a PhosphoPlus® Stat1 (Tyr701) Antibody kit (Cell Signaling Technology, Beverly, Mass.). The Daudi cell extracts contain similar amounts of Stat1 based on Western analysis using a Stat1 antibody. However, only extracts treated with any one of (i) a tandem interferon fusion protein with a polyglutamic acid sequence in between (i.e., IFN-E₁₇₅-IFN), (ii) with an interferon fusion protein with polyglutamic acid on both the carboxyl- and the amino-terminal ends (i.e., E₁₇₅-IFN-E₁₇₅), or with (iii) an interferon fusion protein with polyglutamic acid on the amino-terminal side (i.e., E₁₇₅-IFN) were able to stimulate phosphorylation of Stat1 based on Western analysis using a Phospho-Stat1 (Tyr701) antibody. A control sample treated with polyglutamic acid without interferon sequence does not stimulate phosphorylation of Stat1.

(ii) Assaying the Activity of a Recombinantly Produced GCSF-Polyanionic Polymer

Dimethyl sulphoxide (Me₂SO) can induce neutrophilic differentiation of promyelocytic leukemia HL-60 cells. GCSF can potentiate this neutrophilic differentiation process in Me₂SO treated HL-60 cells via activation of transcription factor STAT3 by the Janus kinase signal transducer JAK2, though GCSF by itself has no effect on HL-60 differentiation (Yamaguchi et al., J Biol Chem; 274: 15575-15581, 1999). A method to assess the activity of GCSF or polyglutamic acid-GCSF is therefore to assay its potency to stimulate phosphorylation of STAT3 in differentiated HL-60 cells.

1-ml cultures of arabinose-induced Top10 strain (Invitrogen, Carlsbad, Calif.) E. coli expressing polyglutamic acid-GCSF from pE175GCSF and expressing polyglutamic acid from pBDUV3B as a negative control were spun down and lysed using 100 μl aliquots of BugBuster™ (Novagen, Madison, Wis.) followed by treatment with Benzonase™ nuclease (Novagen, Madison, Wis.). After centrifugation, 25 μl aliquots from the supernatant fraction were applied to 1-ml aliquots of differentiated HL-60 cells. For the preparation of purified polyglutamic acid-GCSF, 100 ml culture of arabinose-induced Top10 strain (Invitrogen, Carlsbad, Calif.) E. coli expressing polyglutamic acid-GCSF from pE175GCSF was spun down and lysed using 10 ml of BugBuster™ (Novagen, Madison. WI) followed by treatment with Benzonase™ nuclease (Novagen, Madison, Wis.). After centrifugation, the supernatant fraction was diluted 4 fold with 10 mM Tris.HCl pH 7.5 and 1 mM EDTA (TE) and NaCl was added to a final concentration of 0.3 M. The entire sample was then loaded onto a 2-ml DEAE-Sephacel (Amersham Pharmacia Biotech, Piscataway, N.J.) column equilibrated with TE+0.3 M NaCl. After extensive wash with TE+0.3 M NaCl, the column was eluted with TE+0.6 M NaCl and collected as 1-ml fractions. Western analysis using an anti-GCSF antibody (R&D Systems, Minneapolis, Minn.) showed most polyglutamic acid-GCSF were found within the first few fractions after the TE+0.6 M NaCl elution. These fractions were pooled and 25 to 200 μl aliquots were used for assays. Supernatant from EB293 cells (Invitrogen, Carlsbad, Calif.) overexpressing GCSF (Todaro et al., U.S. Pat. No. 6,171,824) and commercially available recombinant GCSF (R&D Systems, Minneapolis, Minn.) were also used as positive controls for the STAT3 phosphorylation assays. For the preparation of HL-60 cells for assay, HL-60 cells were plated in RPMI-1640 media containing 1.25% DMSO, 10% FBS at 2.5×10⁵ Cells/ml. For each assay, 5 mis of cells were plated and grown for 24 hrs. To remove the serum prior to assay, cells were spun down and resuspended into 5 ml 1640 media containing 1.25% DMSO, 0% FBS, and were grown for another 24 hrs. Cells were then spun and resuspended in 1 ml RPMI-1640 media with no serum. Cells were then incubated at 37° C. for 30 min after addition of various forms of polyglutamic acid-GCSF and controls. Cells were spun down and lysed in NP-40 lysis buffer containing protease inhibitors and sodium vanadate. The protein concentration of each soluble lysate was determined by using a BCA assay (Pierce Chemical, Rockford, Ill.). 10-15 μg of lysates were then run on 4-20% Tris-Glycine-SDS gels (invitrogen, Carlsbad, Calif.) and followed by transfer to nitrocellulose membrane for western analysis. Blots were probed and developed with a PhosphoPlus® STAT3 (Tyr705) antibody kit (Cell Signaling Technology, Beverly, Mass.). Samples expressing or containing polyglutamic acid-GCSF or GCSF stimulate STAT3 phosphorylation in Me₂SO treated HL-60 cells. Similar to control HL-60 cells with or without Me₂SO treatment, sample expressing polyglutamic acid only does not stimulate STAT3 phosphorylation in Me₂SO treated HL-60 cells. These data show that polyglutamic acid-GCSF is biologically active and that the presence of polyglutamic acid in the N-terminal region of GCSF does not perturb its biological function.

TABLE 1
Oligonucleotide names and sequences

| | | |
|---|---|---|
| SEQ ID | | |
| NO.: | Oligonucleotide | Nucleotide sequence (5′ to 3′ orientation) |

| | | |
|---|---|---|
| 1 | oPG5F | GAAGAGGAAGAAGAGGAGGAAGAAGAAGAG |
| 2 | oPG5R | TTCCTCTTCTTCTTCCTCCTCTTCTTCCTC |
| 3 | oPG6F | CTATAAAATGCCGGAAGAG |
| 4 | oPG6R | TTCCTCTTCCGGCATTTTATAGAGCT |
| 5 | oPG8F | GAAGAGGAGTAGTCTTCTAACTGCA |
| 6 | oPG8R | GTTAGAAGACTACTCCTC |
| 7 | oGFP-2F | CTAGAGGAACTAGTGGTACCGTAGAAAAAATG |
| 8 | oGFP-2R | ATGGTAGTCGACCGGCGCTGCAGTTGGATCCATTATTTG |
| 9 | oGFP-4F | GCAGCTGAATTCGAGCTTGGTACCGTAG |
| 10 | oDP1F | GGCATGGATGAGCTCTATAAAACCATGGAAGAG |
| 11 | oDP1R | CTGAGATGAGTTTTTGTTCTAGAAAG |
| 12 | oPG9F | GGAGGAAGAGGAGGAAGAGGAAGA |
| 13 | oPG9R | CTCCTCTTCCTCTTCCTCCTCTTC |
| 14 | oPG10F | GGAGTAGTCTTCTAACTGCA |
| 15 | oPG11R | GTTAGAAGACTA |
| 16 | oIFN-3F | GCATCAGTACATGTGTGATCTGCCTCAAACCCAC |
| 17 | oIFN-4R | GTCATTTCTAGAGGCGGAGTTATTATTCTTTACTTCTTCTTAAAC |
| 18 | oMCS1F | GATCCTACCTGACGCTTTTTATCGCAACTCTCT |
| 19 | oMCS1R | CAGTAGAGAGTTGCGATAAAAAGCGTCAGGTAG |
| 20 | oMCS2F | ACTGTTTCTCCATACCCGTTTTTTTGGGCTAAC |
| 21 | oMCS2R | TCCTGTTAGCCCAAAAAAACGGGTATGGAGAAA |
| 22 | oMCS3F | AGGAGGTTAATTAAATGTGCAGACCTGC |
| 23 | oMCS3R | CATGGCAGGTCTGCACATTTAATTAACC |
| 24 | oIFNMCS-3F | GCATCATTAATTAAATGTGTGATCTGCCTCAAACCCACAGC |
| 25 | oIFNMCS-2R | GCATTGGTGCAGTCTAGAAGTTATTACTCCTTACTTCTTAAAC |
| 26 | oIFNBB-1F | TACGACGAAGACACGGAGTGTGATCTGCCTCAAACCCACAGC |
| 27 | oIFNPS-2R | TACGACCTGCAGATTATTCCTTACTTCTTAAACTTTCTTGCAAG |
| 28 | oGCSF-3F | AGGAGGTTAATTAAATGCCATTGGGTCCAGCTAGCTCTCTGCCACAG |
| 29 | oGCSF-3R | TCAATGGTGCAGATCATGTCTGGATCCTCGGGCTGGGC |
| 30 | oGCSF 4F | GTCTCCGAAGACGAGGAGACTCCGCTGGGTCCAGCTAGCTC |
| 31 | oGCSF 4R | TCATGTATGCATGTGCAGATTAAGGCTGGGCAAGGTGGCGTAG |
| 32 | oEDAUG1F | CTACAAAATGCCG |
| 33 | oEDAUG1R | TTCCGGCATTTTGTAGAGCT |
| 34 | oEDTAA1F | GAATAATAGTCTCCTCCTGCACTGCA |
| 35 | oEDTAA1R | GTGCAGGAGGAGACTATTA |

1. A recombinant fusion protein comprising a polyanionic polypeptide and another polypeptide at either one end or at both ends thereof.

2. The recombinant fusion protein according to claim 1, comprising a first polypeptide at the amino-terminal end of said polyanionic polypeptide and a second polypeptide at the carboxyl-terminal end, wherein said first polypeptide and said second polypeptide are the same or are different.

3. The recombinant fusion protein according to claim 2, wherein each of said first polypeptide and said second polypeptide is selected from the group consisting of a targeting polypeptide and a therapeutic polypeptide.

4. The recombinant fusion protein according to claim 3, wherein said first polypeptide and said second polypeptide are different.

5. The recombinant fusion protein according to claim 1, wherein the other polypeptide is selected from the group consisting of an interferon, interferon-α, and granulocyte colony stimulating factor.

6. The recombinant fusion protein according to claim 1, wherein the other polypeptide is an anti-angiogenic protein selected from the group consisting of a pigment epithelium-derived factor, vascular endothelial growth inhibitor, the domain 5 region of high molecular weight kininogen, endostatin, restin, plasminogen kringle 1 domain, plasminogen kringle 5 domain, and angiostatin.

7. The recombinant fusion protein according to claim 1, wherein the other polypeptide is a recognition motif, selected from the group consisting of an antibody, an antibody fragment, folate, AGCKNFFWKTFTSC, ALNGREESP, CNGRC, ATWLPPR and CTTHWGFTLC.

8. The recombinant fusion protein according to claim 1, wherein the polyanionic polymer is polyglutamic acid or polyaspartic acid and the other protein is interferon-α.

9. (canceled)

10. (canceled)

11. The recombinant fusion protein according to claim 1, wherein the polyanionic polymer is polyglutamic acid or polyaspartic acid and the other protein is granulocyte colony stimulating factor.

12-14. (canceled)

15. The recombinant fusion protein according to claim 1, further comprising a spacer amino acid, selected from the group consisting of glycine, an alanine, a β-alanine, a glutamate and leucine.

16-48. (canceled)

49. A method for treating a disease or ailment in an individual comprising administering to said individual an effective amount of a recombinantly-produced polyanionic polymer conjugate or fusion protein, wherein the polyanionic polymer is either polyglutamic acid or polyaspartic acid.

50. The method according to claim 49, wherein the recombinantly-produced polyanionic polymer is conjugated to interferon-α or granulocyte colony stimulating factor.

51. The method according to claim 49, wherein the recombinantly-produced polyanionic polymer is recombinantly linked to any one of interferon-α or, granulocyte colony stimulating factor.

52. The method according to claim 49, wherein the polyanionic polymer further comprises at least one spacer selected from the group consisting of a glycine, an alanine, a P-alanine, a glutamate, leucine, or an isoleucine, diols, aminothiols, hydroxythiols, aminoalcohols, and an spacer comprising the formula, —[NH—(CHR′)p-CO]n-, wherein R′ is a side chain of a naturally occurring amino acid, n is an integer between 1 and 10, most preferably between 1 and 3; and p is an integer between 1 and 10, most preferably between 1 and 3; hydroxyacids of the general formula —[O—(CHR′)p-CO]n-, wherein R′ is a side chain of a naturally occurring amino acid, n is an integer between 1 and 10, most preferably between 1 and 3; and p is an integer between 1 and 10, most preferably between 1 and 3.

53. The method according to claim 49, wherein the polyanionic polymer is joined to interferon-a.

54. (canceled)

55. (canceled)

56. The method according to claim 49, wherein the polyanionic polymer is joined to granulocyte colony stimulating factor.

57-61. (canceled)

62. The recombinant fusion protein according to claim 1, wherein said polyanionic polypeptide is larger than 10 kD.
